(19) United States
(12) Patent Application Publication
(10) Pub. No.: US 2025/0161882 A1
(43) Pub. Date: May 22, 2025

(54) FLUIDIC DEVICES, NANOPORE INSTRUMENTS, AND METHODS

(71) Applicant: ILLUMINA, INC., San Diego, CA (US)

(72) Inventors: Yuliia Vyborna, Sawston (GB); Istvan Kocsis, Cambridge (GB); Alexandre Richez, Cambridge (GB); Davide Garoldini, Cambridge (GB)

(21) Appl. No.: 18/947,506

(22) Filed: Nov. 14, 2024

Related U.S. Application Data
(60) Provisional application No. 63/600,464, filed on Nov. 17, 2023.

(51) Int. Cl. B01D 67/00; B01L 3/00

(52) U.S. Cl. CPC B01D 67/0079; B01L 3/502; B01L 2300/0645

(57) ABSTRACT

An example of a fluidic device includes a support structure, which defines a well and an interstitial region surrounding the well. An electrode is operatively positioned at a bottom of the well. Membrane fragments are bound over the interstitial region, and a nanopore, a nanopore subunit, or a combination thereof is bound over the interstitial region. The fluidic device may be incorporated into a nanopore instrument.

### CROSS-REFERENCE TO RELATED APPLICATION

This application claims the benefit of U.S. Provisional Application Ser. No. 63/600,464, filed Nov. 17, 2023, the contents of which is incorporated by reference herein in its entirety.

### FIELD

This application relates to fluidic devices, nanopore instruments, and methods.

### BACKGROUND

A significant amount of academic and corporate time and energy has been invested into using nanopores for a variety of applications, including to sequence polynucleotides, to identify or quantify proteins, to detect analytes, etc.

However, previously known devices, systems, and methods that utilize nanopores may not necessarily be sufficiently robust, reproducible, or sensitive, and may not have sufficiently high throughput for practical implementation, e.g., demanding commercial applications, such as genome sequencing in clinical and other settings, that demand cost effective and highly accurate operation.

### SUMMARY

Fluidic devices and nanopore instruments are disclosed herein that are suitable for long-term storage and shipping. These fluidic devices and nanopore instruments include support structures with at least one well defined therein. These fluidic devices and nanopore instruments also include nanopores and/or subunits thereof that are temporarily bound to membrane fragments, at least some of which are bound over interstitial regions of the support structure rather than suspended across the aperture of the well. By “bound over,” it is meant that the nanopore and/or nanopore subunit is non-specifically bound to the membrane fragments or is present in a puddle of fluid over the interstitial regions. The nanopore(s) and/or nanopore subunit(s) may also be present in a puddle of fluid over other surfaces of the support structure. The membrane fragments are less fragile than suspended membranes, and thus are more stable when exposed to mechanical stress and/or temperature variation that can take place during storage and/or shipping. Moreover, the membrane fragments present in the device or instrument can be used for membrane repainting over the aperture(s). The temporarily bound nanopores can be reinserted and/or nanopore subunits can be reassembled into a nanopore and reinserted into the newly suspended membranes prior to use in sequencing or another operation.

### BRIEF DESCRIPTION OF DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 is a schematic illustration of a portion of one example of a fluidic device;

FIG. 2 is a schematic illustration of one example of a nanopore instrument including the portion of the fluidic device of FIG. 1, where a trans well of the fluidic device is in fluid communication with a single cis well, and the instrument is shown after a membrane and nanopore of the fluidic device have been introduced at an aperture of the trans well;

FIG. 3 is a schematic illustration of another example of a nanopore instrument including the portion of the fluidic device of FIG. 1, where several trans wells of the fluidic device are in fluid communication with a single cis well, and the instrument is shown before membranes and nanopores of the fluidic device have been introduced at respective apertures of the trans wells;

FIG. 4A schematically illustrates a membrane having a bilayer structure formed with a diblock copolymer, which may be included in the fluidic device and nanopore instruments of FIG. 1, FIG. 2, and FIG. 3;

FIG. 4B schematically illustrates a membrane having a bilayer structure formed with a triblock copolymer, which may be included in the fluidic device and nanopore instruments of FIG. 1, FIG. 2, and FIG. 3;

FIG. 4C schematically illustrates a membrane having a single layer structure formed with the triblock copolymer, which may be included in the fluidic device and nanopore instruments of FIG. 1, FIG. 2, and FIG. 3;

FIG. 5A through FIG. 5E schematically illustrate example schemes for preparing triblock copolymers for use in the fluidic device and nanopore instruments of FIG. 1, FIG. 2, and FIG. 3;

FIG. 6A though FIG. 6C together schematically illustrate an example of a method for preparing an example of the fluidic device for shipping and/or storage, where FIG. 6A depicts the initial formation of a membrane over an aperture of a well, FIG. 6B depicts the initial insertion of a nanopore in the membrane, and FIG. 6C depicts the formation of membrane fragments over the interstitial regions and the nanopore(s) bound over the interstitial regions;

FIG. 7A through FIG. 7C schematically illustrate an example of a method for preparing an example of the fluidic device for use in a sequencing operation, where FIG. 7A depicts the fluidic device with the membrane fragments and nanopore(s) bound over the interstitial regions, FIG. 7B depicts the formation of a suspended membrane over the aperture of the well from the membrane fragments, and FIG. 7C depicts the reinsertion of the nanopore in the suspended membrane;

FIG. 8A and FIG. 8B respectively and schematically illustrate plan and cross-sectional views of further details of the membrane of FIG. 4A and the nanopore inserted therein;

FIG. 9 schematically illustrates a hybrid membrane incorporating molecules of the membrane of FIG. 4B and of FIG. 4C;

FIG. 10 schematically illustrates the nanopore instrument of FIG. 2 during one example of a sequencing operation;

FIG. 11 schematically illustrates the nanopore instrument of FIG. 2 during another example of a sequencing operation;

FIG. 12 schematically illustrates the nanopore instrument of FIG. 2 during still another example of a sequencing operation;

FIG. 13 schematically illustrates the nanopore instrument of FIG. 2 during yet a further example of a sequencing operation;

FIG. 14 schematically illustrates the nanopore instrument of FIG. 2 during an additional example of a sequencing operation;

FIG. 15A and FIG. 15B are graphs depicting current versus voltage curves (IV curves, pA (Y axis) versus mV (X axis)) for 15A: a single pore after it is inserted into an initially formed membrane and 15B: the single pore after it is reinserted to a repainted membrane;

FIG. 16 is a graph depicting the pore stability when exposed to various high voltage pulsing (percentage of pores surviving high voltage pulses, %, Y axis) after initial membrane formation and pore insertion, and for repainted membranes and reinserted pores;

FIG. 17A and FIG. 17B are graphs depicting pore reinsertion efficiency in repainted membranes (percentage of single pores inserted per formed membrane, %, Y axis) for two different fluidic devices exposed to different temperature storage conditions after membrane breakage;

FIG. 18 is a graph depicting pore insertion efficiency (percentage of pores inserted per formed membrane, %, Y axis) for a different fluidic device over time and after multiple membrane repainting and pore insertion events;

FIG. 19 is a graph depicting pore insertion efficiency (percentage of single pores inserted per formed membrane, %, Y axis) for a fluidic device after initial membrane painting and pore insertion and before mechanical stress exposure and after mechanical stress exposure and membrane repainting and pore reinsertion; and

FIG. 20 is a graph depicting DNA capture events for one pore of a fluidic device after membrane formation, breakage, and repainting, and pore reinsertion.

### DETAILED DESCRIPTION

Fluidic devices and nanopore instruments are described herein that exhibit stability during pre-operational processes, such as handling, shipping and/or storage. During pre-operational processes, membrane fragment(s) and nanopores(s) of the fluidic device or nanopore instrument are bound over interstitial regions of a support structure. At some point prior to the operational use of the fluidic device or nanopore instrument (e.g., at the outset of a sequencing operation), the membrane fragment(s) are painted over aperture(s) of well(s) to form suspended membrane(s), and nanopores(s) are moved into an operable position into the suspended membrane(s). Thus, the fluidic devices and nanopore instruments are pre-treated with the membrane fragment(s) and nanopore(s) that will be used in operation, but these components are initially positioned such that the fluidic devices and nanopores instruments are more robust during pre-operational processes.

The example devices and instruments set forth herein can be stored at room temperature (e.g., about 20° C. to about 22° C.) or at ambient temperatures (e.g., from −30° C. to 60° C.) for more than one month, e.g., three or more months, six or more months, one year or more, and then reassembled into an operative position.

In part because of the temperature stability exhibited by the example devices and instruments disclosed herein, the devices and instruments set forth herein can also be shipped without cold packs. During shipping, the devices and instruments can be exposed to extreme low temperatures, such as freezing, and/or extreme high temperatures, such as up to 30° C., or up to 40° C., or up to 60° C.), that may be experienced during normal shipping (e.g., via air, land, and/or sea).

Some of the terms used herein will be briefly explained. Then, some example fluidic devices and nanopore instruments including the membrane fragment(s) and nanopore(s), and methods of making and using the same, will be described.

### Terms

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” is also intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine, or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

As used herein, the term “polynucleotide” refers to a molecule that includes a sequence of nucleotides that are bonded to one another. A polynucleotide is one example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at 3′ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP. Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, DEEP VENTR™ (exo-) DNA polymerase, DEEP VENTR™ DNA polymerase, DYNAZYME™ EXT DNA, DYNAZYME™ II Hot Start DNA Polymerase, PHUSION™ High-Fidelity DNA Polymerase, THERMINATOR™ DNA Polymerase, THERMINATOR™ II DNA Polymerase, VENTR® DNA Polymerase, VENTR® (exo-) DNA Polymerase, REPLIPHI™ Phi29 DNA Polymerase, rBst DNA Polymerase, rBst DNA Polymerase (Large), Fragment (ISOTHERM™ DNA Polymerase), MASTERAMP™ AMPLITHERM™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and ISOPOL™ SD+ polymerase. In specific examples, the polymerase is selected from a group consisting of Bst, Bsu, and Phi29. Some polymerases have an activity that degrades the strand behind them (3′ exonuclease activity). Some useful polymerases have been modified, either by mutation or otherwise, to reduce or eliminate 3′ and/or 5′ exonuclease activity.

Example RNA polymerases include RdRps (RNA dependent, RNA polymerases) that catalyze the synthesis of the RNA strand complementary to a given RNA template. Example RdRps include polioviral 3Dpol, vesicular stomatitis virus L, and hepatitis C virus NS5B protein. Example RNA Reverse Transcriptases include those derived from Avian Myelomatosis Virus (AMV), Murine Moloney Leukemia Virus (MMLV) and/or the Human Immunodeficiency Virus (HIV), telomerase reverse transcriptases such as (hTERT), SUPERSCRIPT™ III, SUPERSCRIPT™ IV Reverse Transcriptase, and PROTOSCRIPT® II Reverse Transcriptase.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-guanine, which may be cleaved under suitable conditions, such as UV light, or upon exposure to chemistry, an enzyme, or another suitable cleaving mechanism. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “plurality” is intended to mean a population of two or more different members. Pluralities may range in size from small, medium, large, to very large. The size of a small plurality may range, for example, from a few members to tens of members. Medium sized pluralities may range, for example, from about tens of members to about hundreds of members. Large pluralities may range, for example, from about hundreds of members to about thousands of members, or up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Accordingly, the definition of the term is intended to include all integer values greater than two.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.”

As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to sample preparation, sequencing, and/or another procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either 3′ end or 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

As used herein, the term “support structure” refers to a material or a combination of materials used as a foundation for supporting composition(s) (e.g., the membrane) described herein. The support structure may be defined out of a single material, or may include a substrate upon which other components (e.g., a patterned layer) of the support structure are positioned.

Example substrate materials may include glass, silica, plastic, quartz, metal, metal oxide, organo-silicate (e.g., polyhedral organic silsesquioxanes (commercially available under the tradename POSS® from Hybrid Plastics)), polyacrylates, tantalum oxide, complementary metal oxide semiconductor (CMOS), or combinations thereof. An example of POSS® can be that described in Kehagias et al., Microelectronic Engineering 86 (2009), pp. 776-778, which is incorporated by reference in its entirety. In some examples, the substrate is a silica-based substrate, such as glass, fused silica, or other silica-containing material. In some examples, silica-based substrates can include silicon, silicon dioxide, silicon nitride, or silicone hydride. In other examples, the substrate is a polymer or includes a polymeric component, such as polyethylene, polystyrene, poly(vinyl chloride), polypropylene, polyamides (i.e., nylons), polyimide, polyesters, polycarbonates, and poly(methyl methacrylate). Example polymeric materials include poly(methyl methacrylate), polystyrene, and cyclic olefin polymer substrates. In some examples, the substrate is or includes a silica-based material or a polymeric material or a combination thereof. In particular examples, the substrate has at least one surface including glass or a silicon-based polymer. In some examples, the substrate can include a metal, such as gold. In some examples, the substrate has at least one surface including a metal oxide. In one example, the surface includes a tantalum oxide or tin oxide. Acrylamides, enones, or acrylates may also be utilized as a substrate material or component. Other example substrate materials include gallium arsenide (GaAs), indium tin oxide (ITO), indium phosphide, aluminum, ceramics, quartz, resins, or other polymers and copolymers. In some examples, the substrate and/or the substrate surface can be, or include, quartz. In some other examples, the substrate and/or the substrate surface can be, or include, a semiconductor, such as GaAs or ITO. The foregoing lists are intended to be illustrative, but not limiting, of the present application. Substrates can include a single material or a plurality of different materials. Substrates can be composites or laminates. In some examples, the substrate includes an organo-silicate material.

The patterned layer of the support structure may be of biological origin, or may be synthetic. As some examples, the patterned layer may include, or may consist essentially of, an organic material, e.g., a curable resin such as SU-8; polytetrafluoroethylene (PTFE), poly(methyl methacrylate) (PMMA), parylene, or the like. Additionally, or alternatively, in various examples, the patterned layer may include, or may consist essentially of, an inorganic material, e.g., silicon nitride, silicon oxide, or molybdenum disulfide.

The support structure can suspend a membrane. The support structure may also define an aperture, such that a first portion of the membrane is suspended across the aperture, and a second portion of the membrane is disposed on, and supported by, the support structure. The support structure may include any suitable arrangement of elements to define the aperture and suspend the membrane across the aperture. As mentioned, the support structure may include the patterned layer, which may have the aperture defined therein, which opens to a well that is also defined therein. Additionally, or alternatively, the patterned layer of the support structure may include one or more first features (such as one or more lips or ledges of the well) that are raised relative to one or more second features (such as a bottom surface of the well), wherein a height difference between (a) the one or more first features and (b) the one or more second features defines the well and the aperture across which the membrane may be suspended. The aperture may have any suitable shape, such as a circle, an oval, a polygon, or an irregular shape.

In some examples, the support structure described herein forms at least part of a flow cell, or is located in or coupled to a flow cell. One example of a flow cell includes a single flow channel (e.g., a single cis well in fluid communication with a plurality of trans wells). Another example of a flow cell includes multiple flow channels (e.g., separate cis wells that are in respective fluid communication with separate sets of trans wells). Example flow cells and support structures for manufacture of flow cells that can be used in methods and compositions set forth herein include, but are not limited to, those commercially available from Illumina, Inc. (San Diego, CA).

As used herein, the term “electrode” is intended to mean a solid structure that conducts electricity. Electrodes may include any suitable electrically conductive material, such as gold, palladium, silver, silver/silver chloride, platinum, or combinations thereof. In some examples, an electrode may be disposed on a substrate. In some examples, an electrode may be a component of a support structure.

As used herein, the term “nanopore” is intended to mean a structure that includes an aperture that permits molecules to cross therethrough from a first side of the nanopore to a second side of the nanopore, in which a portion of the aperture of a nanopore has a width of 100 nm or less, e.g., 10 nm or less, or 2 nm or less. The aperture extends through the first and second sides of the nanopore. Molecules that can cross through an aperture of a nanopore can include, for example, ions or water-soluble molecules, such as amino acids or nucleotides. The nanopore can be disposed within a barrier, or can be provided through a substrate. Optionally, a portion of the aperture can be narrower than one or both of the first and second sides of the nanopore, in which case that portion of the aperture can be referred to as a “constriction.” Alternatively or additionally, the aperture of a nanopore, or the constriction of a nanopore (if present), or both, can be greater than 0.1 nm, 0.5 nm, 1 nm, 10 nm or more. A nanopore can include multiple constrictions, e.g., at least two, or three, or four, or five, or more than five constrictions. Nanopores include biological nanopores or hybrid nanopores.

Biological nanopores include, for example, polypeptide nanopores and polynucleotide nanopores. A “polypeptide nanopore” is intended to mean a nanopore that is made from one or more polypeptides. The one or more polypeptides can include a monomer, a homopolymer, or a heteropolymer. Structures of polypeptide nanopores include, for example, an α-helix bundle nanopore and a β-barrel nanopore as well as all others well known in the art. Example polypeptide nanopores include aerolysin, α-hemolysin, Mycobacterium smegmatis porin A (MspA), gramicidin A, maltoporin, OmpF, OmpC, PhoE, Tsx, F-pilus, SP1, mitochondrial porin (VDAC), Tom40, outer membrane phospholipase A, CsgG, CsgG-CsgF complex, and Neisseria autotransporter lipoprotein (NalP). Mycobacterium smegmatis porin A (MspA) is a membrane porin produced by Mycobacteria, allowing hydrophilic molecules to enter the bacterium. MspA forms a tightly interconnected octamer and transmembrane beta-barrel that resembles a goblet and includes a central constriction. For further details regarding α-hemolysin, see U.S. Pat. No. 6,015,714, the entire contents of which are incorporated by reference herein. For further details regarding SP1, see Wang et al., Chem. Commun., 49:1741-1743 (2013), the entire contents of which are incorporated by reference herein. For further details regarding MspA, see Butler et al., “Single-molecule DNA detection with an engineered MspA protein nanopore,” Proc. Natl. Acad. Sci. 105: 20647-20652 (2008) and Derrington et al., “Nanopore DNA sequencing with MspA,” Proc. Natl. Acad. Sci. USA, 107:16060-16065 (2010), the entire contents of both of which are incorporated by reference herein. Other nanopores include, for example, the MspA homolog from Nocardia farcinica, and lysenin. For further details regarding lysenin, see PCT Publication No. WO 2013/153359, the entire contents of which are incorporated by reference herein. A “polynucleotide nanopore” is intended to mean a nanopore that is made from one or more nucleic acid polymers. A polynucleotide nanopore can include, for example, a polynucleotide origami.

A “hybrid nanopore” is intended to mean a nanopore that is made from two different materials of biological origin or from materials of both biological and non-biological origins. Materials of biological origin are defined above and include, for example, polypeptides and polynucleotides. One example of a hybrid nanopore that includes two different material of biological origin is a polynucleotide origami-protein nanopore complex. As mentioned, the hybrid nanopore may include materials of both biological and non-biological origins. Solid-state nanopores are one example of a nanopore that is made from one or more materials that are not of biological origin. Examples of solid-state nanopores include silicon nitride (SiN), silicon dioxide (SiO₂), silicon carbide (SiC), hafnium oxide (HfO₂), molybdenum disulfide (MoS₂), hexagonal boron nitride (h-BN), or graphene). Examples of biological and solid-state hybrid nanopores include a polypeptide-solid-state hybrid nanopore and a polynucleotide-solid-state nanopore.

As used herein, the term “nanopore subunits” refers to the units that self-assemble to form some biological nanopores. The units may be protein units or polypeptide units. Example protein nanopores that are composed of multiple protein subunits include MspA, CsgG, CsgG/CsgF, Fragacea-toxin C, Aerolysin, and aHL.

As used herein, a “membrane” is intended to mean a structure that normally inhibits passage of molecules from one side of the membrane to the other side of the membrane. The molecules for which passage is inhibited can include, for example, ions or water soluble molecules such as nucleotides and amino acids. However, if a nanopore is disposed within a membrane, then the aperture of the nanopore may permit passage of molecules from one side of the membrane to the other side of the membrane. As one specific example, if a nanopore is disposed within a membrane, the aperture of the nanopore may permit passage of molecules from one side of the membrane to the other side of the membrane. Membranes suitable for use in the examples disclosed herein include amphiphilic bilayers, including those of biological origin, such as amphiphilic lipid bilayers, or those of synthetic origin, such as amphiphilic polymer bilayers.

As used herein, “of biological origin” refers to material derived from or isolated from a biological environment such as an organism or cell, or a synthetically manufactured version of a biologically available structure.

As used herein, “synthetic” refers to a membrane material that is not of biological origin (e.g., polymeric materials, synthetic phospholipids, or combinations thereof).

As used herein, a “solution” is intended to refer to a homogeneous mixture including two or more substances. In such a mixture, a solute is a substance which is dissolved in another substance referred to as a solvent. A solution may include a single solute, or may include a plurality of solutes. An “aqueous solution” refers to a solution in which the solvent is, or includes, water.

As used herein, a “polymeric membrane” or a “polymer membrane” refers to a synthetic barrier that primarily is composed of a polymer that is not of biological origin. In some examples, a polymeric membrane consists essentially of a polymer that is not of biological origin. An amphiphilic block copolymer is an example of a polymer that is not of biological origin and that may be included in the present membranes. A hydrophobic polymer with ionic end groups is another example of a polymer that is not of biological origin and that may be included in the present membranes.

As used herein, the term “block copolymer” is intended to refer to a polymer having at least a first portion or “block” that includes a first type of monomer, and at least a second portion or “block” that is coupled directly or indirectly to the first portion and includes a second, different type of monomer. The first portion may include a polymer of the first type of monomer, or the second portion may include a polymer of the second type of monomer, or the first portion may include a polymer of the first type of monomer and the second portion may include a polymer of the second type of monomer. In the examples set forth herein, at least one of the blocks is hydrophilic and at least one other of the blocks is hydrophobic. In some examples, the monomers themselves impart the desired hydrophilicity and hydrophobicity. In other examples, the first portion optionally may include an end group with a hydrophilicity that is different than that of the first type of monomer and/or the second portion optionally may include an end group with a hydrophilicity that is different than that of the second type of monomer. The end groups of any hydrophilic blocks may be located at an outer surface of a barrier/membrane formed using such hydrophilic blocks. Depending on the particular configuration, the end groups of any hydrophobic blocks may be located at an inner surface of the barrier or at an outer surface of a barrier formed using such hydrophobic blocks.

Block copolymers include, but are not limited to, diblock copolymers and triblock copolymers.

A “diblock copolymer” is intended to refer to a block copolymer that includes, or consists essentially of, first and second blocks coupled directly or indirectly to one another. The first block may be hydrophilic and the second block may be hydrophobic, in which case the diblock copolymer may be referred to as an “AB” copolymer where “A” refers to the hydrophilic block and “B” refers to the hydrophobic block.

A “triblock copolymer” is intended to refer to a block copolymer that includes, or consists essentially of, first, second, and third blocks coupled directly or indirectly to one another. The first and third blocks may include, or may consist essentially of, the same type of monomer (repeating unit) as one another, and the second block may include a different type of monomer (repeating unit). In other examples, the first block may be hydrophilic, the second block may be hydrophobic, and the third block may be hydrophilic and include the same type of monomer as the first block, in which case the triblock copolymer may be referred to as an “ABA” copolymer where “A” refers to the hydrophilic blocks and “B” refers to the hydrophobic block.

The particular arrangement of molecules of polymer chains (e.g., block copolymers) within a polymeric membrane may depend, among other things, on the respective block lengths, the type(s) of monomers used in the different blocks, the relative hydrophilicities and hydrophobicities of the blocks, the composition of the fluid(s) within which the membrane is formed, and/or the density of the polymeric chains within the membrane. During formation of the membrane, these and other factors generate forces between molecules of the polymeric chains which laterally position and reorient the molecules in such a manner as to substantially minimize the free energy of the membrane. The membrane may be considered to be substantially “stable” once the polymeric chains have completed these rearrangements, even though the molecules may retain some fluidity of movement within the membrane.

As used herein, the term “hydrophobic” is intended to mean tending to exclude water molecules. Hydrophobicity is a relative concept relating to the polarity difference of molecules relative to their environment. Non-polar (hydrophobic) molecules in a polar environment will tend to associate with one another in such a manner as to reduce contact with polar (hydrophilic) molecules to a minimum to lower the free energy of the system as a whole.

As used herein, the term “hydrophilic” is intended to mean tending to bond to water molecules. Polar (hydrophilic) molecules in a polar environment will tend to associate with one another in such a manner as to reduce contact with non-polar (hydrophobic) molecules to a minimum to lower the free energy of the system as a whole.

As used herein, the term “amphiphilic” is intended to mean having both hydrophilic and hydrophobic properties. For example, a block copolymer that includes a hydrophobic block and a hydrophilic block may be considered to be “amphiphilic.” Illustratively, AB copolymers and ABA copolymers all may be considered to be amphiphilic.

As used herein, the term “linker” is intended to mean a moiety, molecule, or molecules via which one element is attached to another element. Linkers may be covalent, or may be non-covalent. Examples of covalent linkers include moieties such as alkyl chains, polyethers, amides, esters, aryl groups, polyaryls, and the like. Examples of noncovalent linkers include host-guest complexation, cyclodextrin/norbornene, adamantane inclusion complexation with β-CD, DNA hybridization interactions, streptavidin/biotin, and the like. In examples of the block copolymers set forth herein, the linker is covalent.

As used herein, the terms “PEO”, “PEG”, “poly(ethylene oxide)”, and “poly(ethylene glycol)” are intended to be used interchangeably and refer to a polymer that comprises —[CH₂—CH₂—O]ₙ—. In some examples, n is between about 2 and about 100.

As used herein, the term “annulus” is intended to refer to a liquid that is adhered to a support structure, located within a membrane, and extends partially into the aperture and well defined by the support structure. As such, it will be understood that the annulus may follow the shape of the membrane, e.g., may have the shape of a circle, an oval, a polygon, or an irregular shape.

The term “operable position” refers to the configuration of the membrane and nanopore when the fluidic device or the nanopore instrument is to be used in a sequencing operation, analyte capture, proteomics, DNA profiling, spatial surveillance, minimal residual disease (MRD) surveillance, or pathogen surveillance. In the examples set forth herein, the operable position of the membrane is when it is suspended over the aperture of a well, and the operable position of the nanopore is when it is inserted into the membrane.

The term “inoperable position” refers to the configuration of the membrane and nanopore when the fluidic device or the nanopore instrument is to be used in pre-operational processes, such as handling, shipping and/or storage. In the examples set forth herein, the inoperable position of the membrane is when it is positioned over the interstitial regions, and the inoperable position of the nanopore is when it is attached to the membrane over the interstitial regions.

### Fluidic Devices and Nanopore Instruments

An example of a portion of a fluidic device 10 is shown in FIG. 1 and includes a support structure 12 defining a well 14 and an interstitial region 16 surrounding the well 14; an electrode 18 operatively positioned at a bottom of the well 14; membrane fragments 19 positioned on the interstitial region 16; and a nanopore 22, a nanopore subunit, or a combination thereof bound over the membrane fragments 19. In FIG. 1, the membrane fragments 19 and the nanopore 22 are shown in the inoperable position. To form the full fluidic device 10, the portion shown in FIG. 1 may include additional structural components that define a flow channel in fluid communication with the well 14. The portion shown in FIG. 1 may be referred to herein as the fluidic device 10.

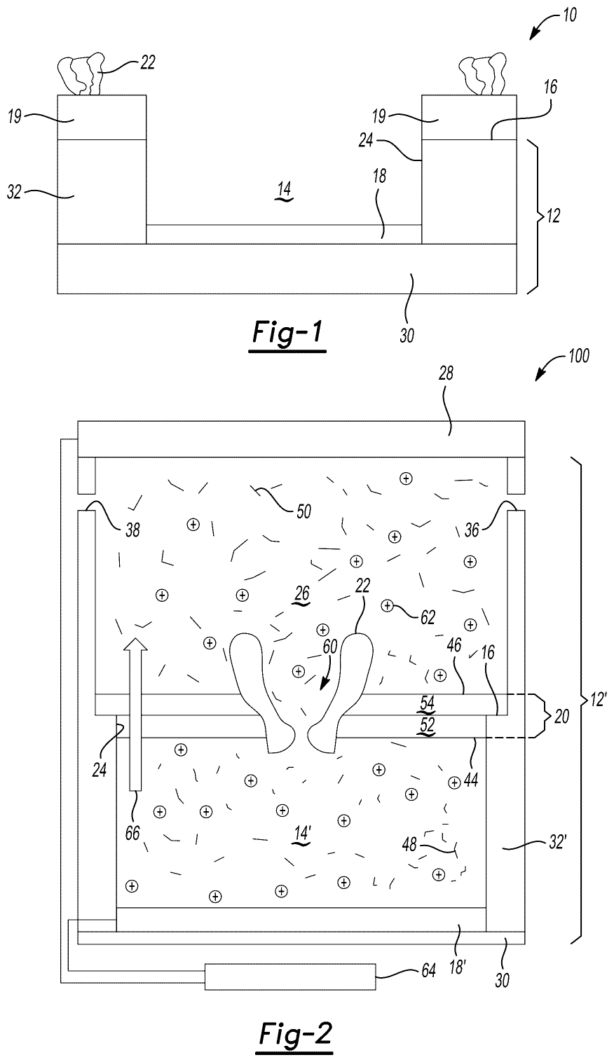

An example of a nanopore instrument 100 is shown in FIG. 2. The nanopore instrument 100 includes the portion of the fluidic device 10 of FIG. 1 incorporated into the instrument 100. The instrument 100 includes the support structure 12′ defining a trans well 14′ and an interstitial region 16 surrounding the trans well 14′; a trans electrode 18′ associated with the trans well 14′; a cis well 26 in fluid communication with the trans well 14′; a cis electrode 28 associated with the cis well 26; a membrane 20; and a nanopore 22 bound to the membrane 20. After initial manufacturing, the membrane 20 is positioned on the interstitial region 16 and over an aperture 24 between the cis and trans wells 26, 14′, and the nanopore 22 is bound to the membrane 20. The membrane 20 is then broken into the membrane fragments 19, which become bound over interstitial region 16. Prior to use in sequencing or another operation, the membrane fragments 19 are painted over the aperture 24 to reform the membrane 20, and the nanopore 22 is repositioned in the membrane 20 or the nanopore subunits are reassembled and repositioned in the membrane 20. The operable position of the membrane fragments 19 (as the membrane 20) and nanopore 22 is shown in FIG. 2.

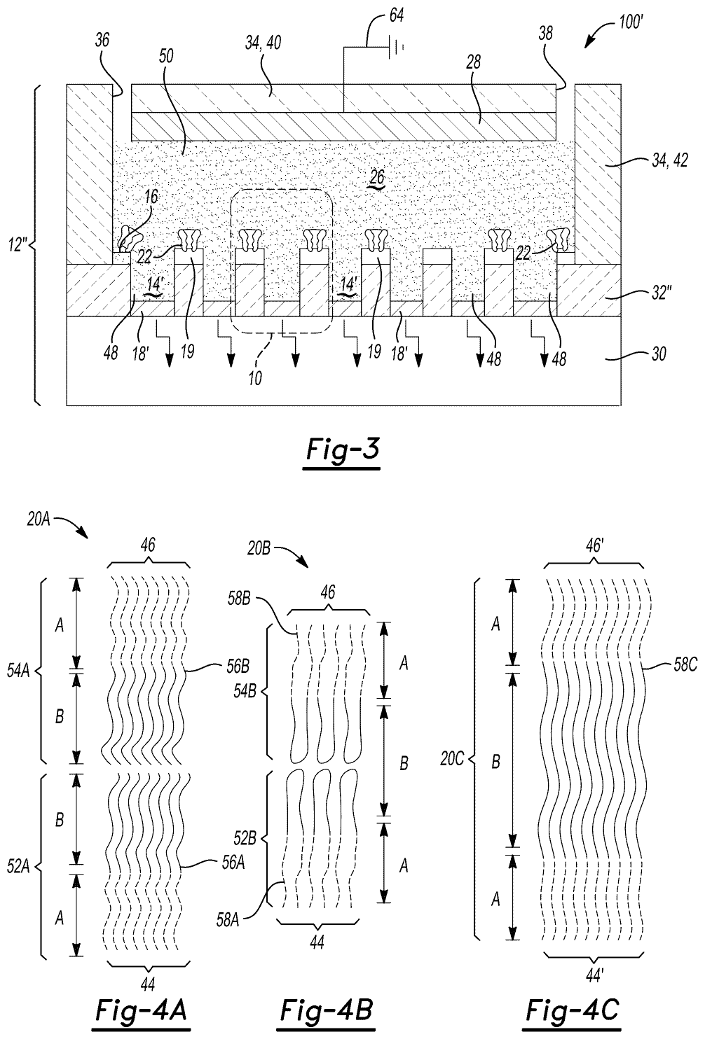

Another example of a nanopore instrument 100′ is shown in FIG. 3 and includes a support structure 12″ defining a plurality of trans wells 14′ and interstitial regions 16 surrounding each of the plurality of trans wells 14′; a trans electrode 18′ associated with the trans wells 14′; a cis well 26 in fluid communication with at least some of the plurality of trans wells 14′; a cis electrode 28 associated with the cis well 26; membrane fragments 19 bound over the interstitial regions 16; and a plurality of nanopores 22 bound over the membrane fragments 19. In one example (as shown in FIG. 3), the instrument 100′ includes a plurality of the trans electrodes 18′, where each of the plurality of the trans electrodes 18′ is respectively associated with one of the plurality of trans wells 14′. In FIG. 3, the membrane fragments 19 and the nanopores 22 are shown in the inoperable position.

In the examples set forth herein, the fluidic device 10 and the nanopore instrument 100, 100′ include the support structure 12, 12′, 12″. In the examples shown in FIG. 1 and FIG. 2, the support structure 12, 12′ includes a substrate 30 and a patterned layer 32, 32′. In the example shown in FIG. 3, the support structure 12″ includes first and second substrates 30, 34 and the patterned layer 32″, which acts as an interposer between the first and second substrates 30, 34.

In the example shown in FIG. 1, the substrate 30 supports the patterned layer 32. In one example, the substrate 30 and the patterned layer 32 are formed as a single piece (e.g., the substrate 30 is patterned with the well 14 and the interstitial regions 16). In another example, the substrate 30 and the patterned layer 32 are separate pieces made of the same material or different materials. In these examples, the substrate 30 and the patterned layer 32 are adhered together.

The substrate 30 may be any of the substrate materials set forth herein. In one example, the substrate 30 may be a semi-conductor (e.g., silicon) wafer.

The patterned layer 32 may be any material that is capable of being processed with nanometer or micrometer accuracy for defining the well 14. In one example, the patterned layer 32 may be formed of a negative photoresist (e.g., an epoxy-based negative photoresist such as SU-8). Other specific examples of the patterned layer 32 include SUEX® (epoxy photoresist from DJ Microlaminates), polyimide, and perylene. The patterned layer 32 (and its features, such as the well(s) 14) may be fabricated using microlithography.

Any suitable securing mechanism may be used to adhere the substrate 30 and the patterned layer 32 together. Some example adhesives include a thermoset or thermoplastic polymer, tape, and a photocurable glue (e.g., acrylic based). Alternatively, the substrate 30 and the patterned layer 32 may be laminated together, e.g., by applying heat and/or mechanical pressure.

The example shown in FIG. 2 is similar to the example shown in FIG. 1, except that the patterned layer 32′ is processed to define both a trans well 14′ and a cis well 26 in fluid communication with the trans well 14′ through an aperture 24 that leads to the trans well 14′. In this example, the patterned layer 32′ also defines interstitial regions 16 that are able to support the membrane fragments 19 or the membrane 20. The interstitial regions 16 are substantially planar surfaces that are adjacent to the aperture 24. The area of the interstitial regions 16 may be sufficient to support the membrane fragments 19 during pre-operational processes and to support the membrane 20 during operation.

As shown in FIG. 2, the patterned layer 32′ has an inlet 36 and an outlet 38 formed therein. The inlet 36 and outlet 38 may be positioned at opposed ends of the cis well 26 or anywhere along the length and width of the cis well 26 that enables desirable fluid flow. While not shown, it is to be understood that the inlet 36 and outlet 38 are fluidly connected to a fluidic control system (including, e.g., reservoirs, pumps, valves, waste containers, and the like) which controls fluid introduction and expulsion.

In the example shown in FIG. 3, the substrate 30 supports the patterned layer 32″ and the second substrate 34. In one example, the substrate 30 and the patterned layer 32″ are formed as a single piece so the substrate 30 defines the wells 14′ and the interstitial regions 16. In another example, the substrate 30 and the patterned layer 32″ are separate pieces made of the same material or different materials. The substrate 30 may be any of the substrate materials set forth herein, and the patterned layer 32″ may be any material set forth herein (e.g., a negative photoresist, an epoxy photoresist, polyimide, or perylene) that is capable of being processed with nanometer or micrometer accuracy for defining the plurality of trans well 14′. In these examples, the substrate 30 and the patterned layer 32″ are adhered together as described herein.

In the example shown in FIG. 3, the second substrate 34 functions as a lid and has the cis well 26 defined therein. The second substrate 34 also has the inlet 36 and outlet 38 defined therein so that fluid may be introduced into and extracted from the cis well 26.

The second substrate 34 may be any of the substrate materials set forth herein, such as polymer, glass, ceramic or metal. The second substrate 34 (and its features, such as the cis well 26, the inlet 36, and the outlet 38) may be fabricated using injection molding or CNC (computer numerical control) machining. It is to be understood that the second substrate 34 may be fabricated monolithically using, for example, additive manufacturing techniques; or the second substrate 34 may be an assembly of parts joined together using appropriate joining technology, including welding and/or adhesives. For example, a sub-assembly defining the top portion 40 may be bonded, welded, or mechanically attached to an interposer (which defines the sidewalls 42) after installation of the membrane 20 and nanopores 22 onto the substrate 30, which has the wells 14′ and interstitial regions 16 defined therein.

The second substrate 34 is a separate piece from the patterned layer 32″, but the two components 32″, 34 may be formed of the same material or different materials. Any of the securing mechanisms described herein may also be used to adhere the second substrate 34 and the patterned layer 32″ together. The substrate 34 is attached to the patterned layer 32″ such that each of the trans wells 14′ is in fluid communication with the cis well 26. While a single array of trans wells 14′ is shown in fluid communication with the one cis well 26, it is to be understood that the nanopore instrument 100′ may include several fluidically isolated cis wells 26, each of which is in fluid communication with a separate array of trans wells 14′. Multiple cis wells 26 may be desirable, for example, in order to enable the measurement of multiple samples using a single nanopore instrument 100′.

Each of the well 14 and the trans well(s) 14′ has an aperture 24 that is defined by the patterned layer 32, 32′, 32″. The aperture 24 defines the opening of the well 14, 14′, and the depth of the well 14, 14′ may extend through the thickness of the patterned layer 32, 32′, 32″. In the examples shown in FIG. 1 through FIG. 3, each trans well 14, 14′ has a sidewall that is defined by the patterned layer 32, 32′, 32″ and a lower surface that is defined by a trans electrode 18.

The trans wells 14, 14′ may be micro wells (having at least one dimension on the micron scale, e.g., about 1 μm up to, but not including, 1000 μm) or nanowells (having at least one dimension on the nanoscale, e.g., about 10 nm up to, but not including, 1000 nm). Each trans well 14, 14′ may be characterized by its aspect ratio (e.g., width or diameter divided by depth or height, respectively).

In an example, the aspect ratio of each trans well 14, 14′ may range from about 1:1 to about 1:5. In another example, the aspect ratio of each trans well 14, 14′ may range from about 1:10 to about 1:50. In an example, the aspect ratio of the trans well 14, 14′ is about 3.3.

The depth/height and width/diameter may be selected in order to obtain a desirable aspect ratio. The depth/height of each trans well 14, 14′ can be at least about 0.1 μm, about 1 μm, about 10 μm, about 100 μm, or more. Alternatively or additionally, the depth can be at most about 1,000 μm, about 100 μm, about 10 μm, about 1 μm, about 0.1 μm, or less. In one example, the depth/height ranges from about 15 μm to about 50 μm. The width/diameter of each trans well 14, 14′ can be at least about 50 nm, about 0.1 μm, about 0.5 μm, about 1 μm, about 10 μm, about 100 μm, or more. Alternatively or additionally, the width/diameter can be at most about 1,000 μm, about 100 μm, about 10 μm, about 1 μm, about 0.5 μm, about 0.1 μm, about 50 nm, or less. In one example, the width/diameter ranges from about 15 μm to about 50 μm.

Each trans well 14, 14′ has the aperture 24 (e.g., that faces the cis well 26 in some examples) that is large enough to accommodate at least a portion of the membrane 20 and the nanopore 22 that is associated therewith when these components are in the operable position. For example, an end of the nanopore 22 may extend through the membrane 20 and into the aperture 24 of the trans well 14, 14′ (as shown in FIG. 2).

When a plurality of the trans wells 14, 14′ are used, many different layouts of multiple trans wells 14, 14′ may be envisaged, including regular, repeating, and non-regular patterns. In an example, the trans wells 14, 14′ are disposed in a hexagonal grid for close packing and improved density. Other layouts may include, for example, rectangular layouts, triangular layouts, and so forth. As examples, the layout or pattern can be an x-y format of trans wells 14, 14′ that are in rows and columns.

The layout may be characterized with respect to the density of the trans wells 14, 14′ (i.e., number of trans wells 14, 14′ in a defined area of the patterned layer 32, 32′, 32″). For example, the trans wells 14, 14′ may be present at a density ranging from about 10 wells per mm² to about 1,000,000 wells per mm². The density may be tuned to different densities including, for example, a density of at least about 10 per mm², about 5,000 per mm², about 10,000 per mm², about 0.1 million per mm², or more. Alternatively or additionally, the density may be tuned to be no more than about 1,000,000 wells per mm², about 0.1 million per mm², about 10,000 per mm², about 5,000 per mm², or less. It is to be further understood that the density of the trans wells 14, 14′ in the patterned layer 32, 32′, 32″ can be between one of the lower values and one of the upper values selected from the ranges above.

The layout may also or alternatively be characterized in terms of the average pitch, i.e., the spacing from the center of a trans well 14, 14′ to the center of an adjacent trans well 14, 14′ (center-to-center spacing). The pattern can be regular such that the coefficient of variation around the average pitch is small, or the pattern can be non-regular in which case the coefficient of variation can be relatively large. In an example, the average pitch may range from about 100 nm to about 500 μm. The average pitch can be, for example, at least about 100 nm, about 5 μm, about 10 μm, about 100 μm, or more. Alternatively or additionally, the average pitch can be, for example, at most about 500 μm, about 100 μm, about 50 μm, about 10 μm, about 5 μm, or less. The average pitch for an example array including a particular pattern of trans wells 14, 14′ can be between one of the lower values and one of the upper values selected from the ranges above. In an example, the array has an average pitch (center-to-center spacing) of about 10 μm.

As shown in each of FIG. 1 through FIG. 3, a trans electrode 18, 18′ is positioned at the bottom of each trans well 14, 14′. A surface of the trans electrode 18, 18′ defines the lower surface of the trans well 14, 14′, and may be physically connected to the patterned layer 32, 32′, 32″. The trans electrode 18, 18′ may be attached to the patterned layer 32, 32′, 32″ during the formation of the trans wells 14, 14′. Microfabrication techniques that may be used to form the trans wells 14, 14′ in the patterned layer 32, 32′, 32″ and to form the trans electrodes 18, 18′ in the trans wells 14, 14′ include lithography, metal deposition and liftoff, dry and/or spin on film deposition, etching, etc. The interface between the trans electrode 18, 18′ and the patterned layer 32, 32′, 32″ may seal the lower portion of the trans well 14, 14′.

The trans electrode 18, 18′ may be any suitable electrode material, such as gold (Au), platinum (Pt), ruthenium (Ru), carbon (C) (e.g., graphite, diamond, etc.), silver/silver chloride (Ag/AgCl) or rhodium (Rh). Layered electrodes, e.g., silver/silver chloride over gold, may also be used. In some examples, the electrodes are passive, in that they do not actively participate in a redox reaction taking place within the device or instrument 100, 100′. In these examples, a redox mediator, such as ferrocyanide/ferricyanide redox couple, is included in an electrolyte buffer in which ferrocyanide ions (e.g., Fe(CN)₆⁴⁻) are oxidized to ferricyanide ions (e.g., Fe(CN)₆³⁻).

When included, the cis well 26 may have any suitable geometry and/or dimensions. In the examples shown in FIG. 2 and FIG. 3, the cis well 26 has a rectangular shaped geometry. The dimensions of the cis well 26 may range from about 1 mm×0.1 mm×0.1 mm to about 100 mm×10 mm×10 mm.

The nanopore instruments 100, 100′ (FIG. 2 and FIG. 3) each include a cis electrode 28. The cis electrode 28 may be any of the examples set forth herein for the trans electrode 18, 18′.

In the example shown in FIG. 2, the cis electrode 28 is attached to a top surface of the patterned layer 32′ so that it forms a top of the cis well 26. In the example shown in FIG. 3, the cis electrode 28 is secured to an interior surface of the substrate 34. The cis electrode 28 may be formed via metal deposition or may be pre-formed and secured to the patterned layer 32′ or substrate 34 using any suitable adhesive.

As illustrated in FIG. 2, the nanopore instruments 100, 100′ include circuitry 64. Generally, the circuitry 64 is in operable communication with the cis and trans electrodes 26, 18′ and is configured to detect changes in an electrical characteristic of the opening 60 of the nanopore 22. Such changes may, for example, be responsive to any suitable stimulus. Indeed, it will be appreciated that the present methods, compositions, and devices may be used in any suitable application or context, including any suitable method or device for polynucleotide sequencing.

The circuitry 64 may be integrated into a semi-conductive wafer that functions as or is in contact with the substrate 30. The circuitry 64 includes at least a stimulus source and a controller (not shown). The stimulus source may be coupled to each trans electrode 18′ either individually or via multiplexing, and the stimulus source is to cause current to flow through one or more of the nanopores 22 by addressing the trans electrode(s) 18′ associated with a respective nanopore 22. The controller is coupled to the stimulus source, and the controller is configured to individually/selectively address one of the electrode(s) 18′ (using the stimulus source) to cause an ionic current to flow through the nanopore 22 connected to the addressed electrode 18′. In one example, each electrode 18′ is electrically connected to its own set of electronics, which includes the stimulus source and the controller. In another example, each of the electrodes 18′ is electrically connected to a single stimulus source and controller, which are connected to a multiplexer. The circuitry 64 may also include operational amplifier(s) to amplify electrical signals passing through respective nanopores 22 associated with electrodes 18′ that are addressed.

The fluidic device 10 shown in FIG. 1 may also include circuitry suitable for the operational use of the device 10.

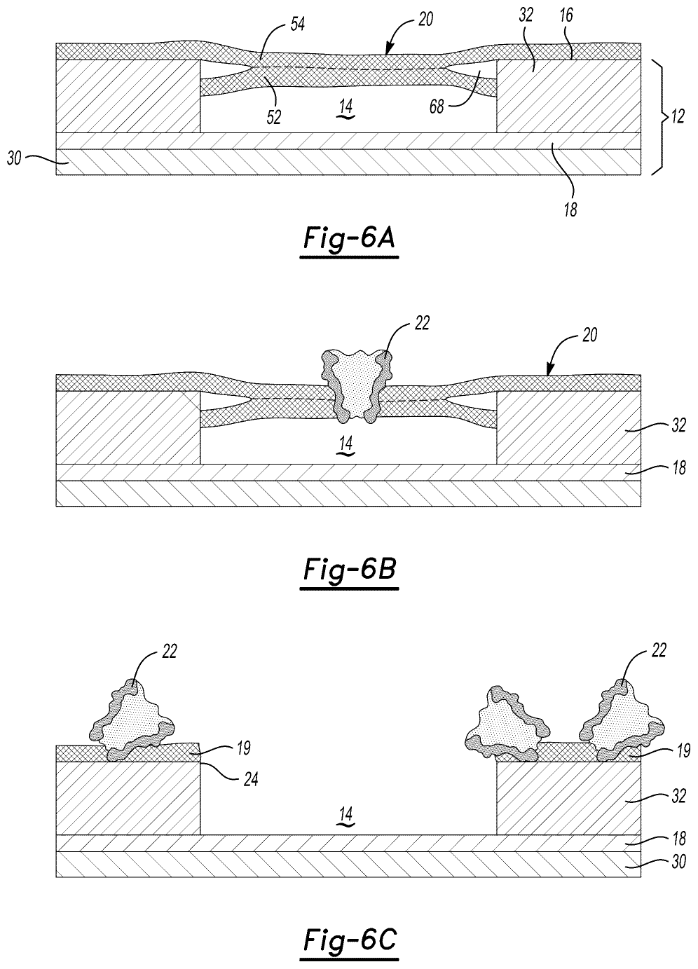

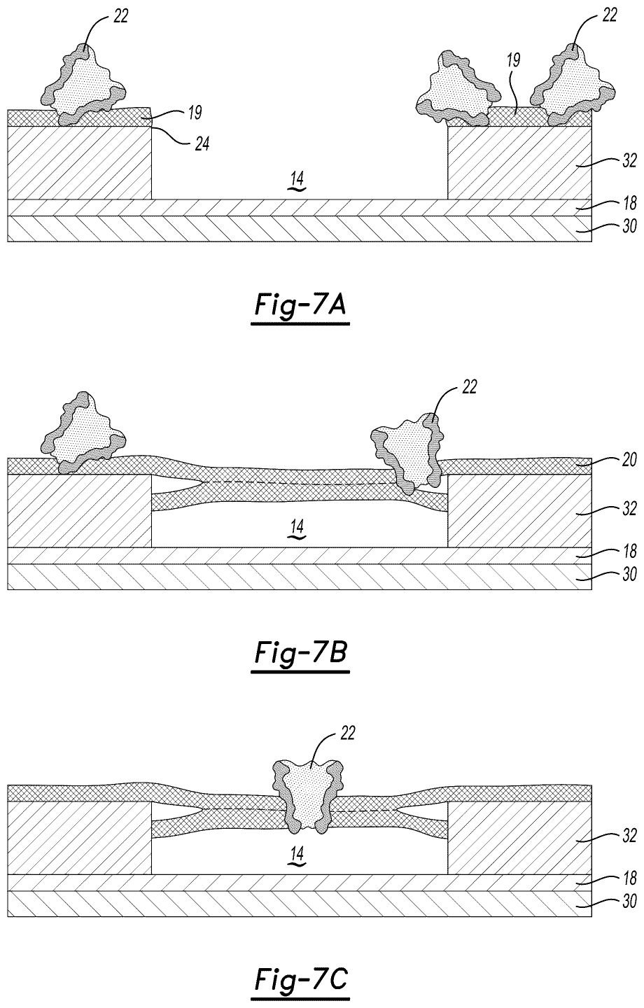

The fluidic device 10 and nanopore instruments 100, 100′ include the membrane fragments 19 in the inoperative position or the membrane 20 in the operative position, depending upon which position the device 10 or instrument 100, 100′ is in. As will be described in further detail in reference to the methods (see FIG. 6A through FIG. 6C and FIG. 7A through FIG. 7C), the membrane fragments 19 are able to be moved from the inoperable position (FIG. 1, FIG. 3, FIG. 6C, and FIG. 7A) to form the membrane 20 (FIG. 2, FIG. 6B, FIG. 7B, and FIG. 7C).

As shown in FIG. 2, when in the operable position across the aperture 24, the membrane 20 has a first (trans) side 44 facing the trans well 14′ and a second (cis) side 46 facing the cis well 26. The membrane 20 may have any suitable structure that normally inhibits passage of molecules from one side of the membrane 20 to the other side of the membrane 20, e.g., that normally inhibits contact between fluids 48, 50 contained in the respective wells 14′, 26. For example, as illustrated in FIG. 2, the structure of the membrane 20 may be a bilayer structure including a first layer 52 and a second layer 54, one or both of which inhibit(s) the flow of molecules across the respective layer. The first and second layers 52, 54 may be formed using examples of the AB diblock copolymers provided herein, or certain ABA triblock copolymers provided herein, and may have a structure as described in greater detail in reference to FIG. 4A and FIG. 4B. Alternatively, the structure of the membrane 20 may include a single layered structure, which inhibits the flow of molecules across that layer. The single layered membrane 20 may be formed using certain ABA triblock copolymers provided herein, and may have a structure such as described in greater detail below with reference to FIG. 4C. In still other examples, the structure of the membrane 20 may be partially a single layer, and partially a bilayer, formed using certain ABA triblock copolymers provided herein.

When the membrane 20 has a bilayer structure, the first layer 52 includes a first plurality of molecules of a diblock or triblock copolymer, and the second layer 54 includes a second plurality of molecules of the copolymer. In examples in which the copolymer is a diblock copolymer (which may be referred to as AB), each molecule may include a hydrophobic block coupled to a hydrophilic block. The hydrophilic blocks of the first plurality of molecules may form a first outer surface of membrane 20, e.g., the surface of layer 52 forming the first side 44. The hydrophilic blocks of the second plurality of molecules may form a second outer surface of membrane 20, e.g., the surface of layer 54 forming the second side 46.

The hydrophobic blocks of the first and second pluralities of molecules may contact one another within the membrane 20. In examples in which the copolymer is a triblock copolymer (which may be referred to as ABA), each molecule may include first and second hydrophilic blocks and a hydrophobic block disposed therebetween. In the bilayer structure, each layer 52, 54 is composed of folded ABA molecules. More particularly, the B blocks of each of the first plurality of molecules are folded such that both A blocks of each molecule form a first outer surface of membrane 20, e.g., the surface of layer 52 forming the first side 44. Similarly, the B blocks of each of the second plurality of molecules are folded such that both A blocks of each molecule form a second outer surface of membrane 20, e.g., the surface of layer 54 forming the second side 46. The folded hydrophobic B blocks of the first and second plurality of molecules may contact one another within the membrane 20.

When the membrane 20 has a single layered structure, the single layer includes a plurality of molecules of the triblock copolymer. In this example, the ABA molecules extend from the trans well 14′ to the cis well 26, forming surfaces 44, 46 (shown as 44′, 46′ in FIG. 4C).

Referring now to FIG. 4A, the bilayer membrane 20A is formed of a diblock “AB” copolymer. The membrane 20A includes the first layer 52A which may contact the fluid 48 in the trans well 14′ and the second layer 54A which may contact the fluid 50 in the cis well 26. The first layer 52A includes a first plurality of molecules 56A of a diblock AB copolymer, and the second layer 54A includes a second plurality of the molecules 56B of the diblock AB copolymer. As illustrated in FIG. 4A, each molecule 56A, 56B of the diblock copolymer includes a hydrophobic block, denoted “B” and being approximately of length “B,” coupled to a hydrophilic block, denoted “A” and being approximately of length “A”. The hydrophilic blocks A of the first plurality of molecules 56A (the molecules forming layer 52A) form the first outer surface 44 of the membrane 20A. The hydrophilic blocks A of the second plurality of molecules 56B (the molecules forming layer 54A) form the second outer surface 46 of the membrane 20A. The respective ends of the hydrophobic blocks B of the first and second pluralities of molecules 56A, 56B contact one another within the membrane 20A in a manner such as illustrated in FIG. 4A. As illustrated, substantially all of the molecules 56A within layer 52A may extend substantially linearly and in the same orientation as one another, and similarly, substantially all of the molecules 56B within layer 54A may extend substantially linearly and in the same orientation as one another (which is opposite that of the orientation the molecules 56A within layer 52A). Accordingly, first and second layers 52A, 54A each may have a thickness of approximately A+B, and the membrane 20A may have a thickness of approximately 2A+2B. In some examples, length A is about 2 repeating units (RU) to about 100 RU, or about 1 repeating unit (RU) to about 50 RU, e.g., about 5 RU to about 40 RU, or about 10 RU to about 30 RU, or about 10 RU to about 20 RU, or about 20 RU to about 40 RU. Additionally, or alternatively, in some examples, length B is about 2 RU to about 100 RU, 5 RU to about 100 RU, e.g., about 10 RU to about 80 RU, or about 20 RU to about 50 RU, or about 50 RU to about 80 RU.

Referring now to FIG. 4B, the bilayer membrane 20B is formed of a triblock “ABA” copolymer. The membrane 20B includes the first layer 52B which may contact the fluid 48 in the trans well 14′ and the second layer 54B which may contact the fluid 50 in the cis well 26. The first layer 52B includes a first plurality of molecules 58A of a triblock ABA copolymer, and the second layer 54B includes a second plurality of the molecules 58B of the triblock ABA copolymer. As illustrated in FIG. 4B, the hydrophobic block, denoted “B,” of each molecule 58A, 58B folds over so that the hydrophilic blocks, denoted “A” and being approximately of length “A,” i) of the molecules 58A form the surface 44 and ii) of the molecules 58B form the surface 46. The folded ends of the hydrophobic blocks B of the first and second pluralities of molecules 58A, 58B contact one another within the membrane 20B in a manner such as illustrated in FIG. 4B. Together, the folded ends of the hydrophobic blocks B are approximately of length “B.”

Referring now to FIG. 4C, the bilayer membrane 20C is also formed of a triblock “ABA” copolymer. The membrane 20C includes a single layer of molecules 58C of the triblock copolymer. As illustrated in FIG. 4C, each molecule 58C of the triblock copolymer includes first and second hydrophilic blocks, each denoted “A” and being approximately of length “A,” and a hydrophobic block, denoted “B” and being approximately of length “B,” disposed between the first and second hydrophilic blocks A. The hydrophilic blocks A at first ends of molecules 58C form a first outer surface 44′ of the membrane 20C, which, e.g., contacts fluid 48 in the trans well 14′. The hydrophilic blocks A at second ends of molecules 58C form a second outer surface 46′ of the of the membrane 20C, which, e.g., contacts fluid 50 in the cis well 26. The hydrophobic blocks B of the molecules 58C are within the membrane 20C in a manner such as illustrated in FIG. 4C. As illustrated, the molecules 58C extend substantially linearly and in the same orientation as one another.

In still other examples (not shown), some of the molecules, e.g., similar to 58A, 58B, of the triblock copolymer may fold and some other molecules, e.g., similar to 58C, 58D of the triblock copolymer may remain linear. Accordingly, these membranes 20 may be considered to be partially a single layer, and partially a bilayer.

Regardless of whether the membrane 20B or 20C includes molecules 58C that extend substantially linearly and/or molecules 58A, 58B that are folded, the membrane 20B or 20C may have a thickness of approximately 2A+B. In some examples, length A is about 1 RU to about 100 RU, e.g., about 2 RU to about 100 RU, or about 10 RU to about 80 RU, or about 20 RU to about 50 RU, or about 50 RU to about 80 RU. Additionally, or alternatively, in some examples, length B is about 2 RU to about 100 RU, or about 5 RU to about 100 RU, e.g., about 10 RU to about 80 RU, or about 20 RU to about 50 RU, or about 50 RU to about 80 RU.

It will be appreciated that any end groups that are coupled to the hydrophilic or hydrophobic blocks A, B in the examples disclosed herein contribute to the overall thickness of the membrane 20 (e.g., 20A, 20B, 20C).

It will be further appreciated that the layer(s) of the various membranes 20 provided herein may be configured so as to have any suitable dimensions. Illustratively, the following examples may be used to form membranes 20 of similar dimension as one another:

A-B diblock copolymer (FIG. 4A) may have 1 hydrophilic block of length A (M_{w}=x), and 1 hydrophobic block of length B (M_{w}=y/2); when self-assembled, those A-B diblock copolymers would form membranes with a top hydrophilic layer of length A, a core hydrophobic layer of length 2B, and a bottom hydrophilic layer of length A.

A-B-A triblock copolymer (FIG. 4B and FIG. 4C) may have 2 hydrophilic blocks, each of length A (each A block is of M_{w}=x) and 1 hydrophobic block of length B (M_{w}=y); when self-assembled in either a folded manner or a linear manner, those A-B-A triblock copolymers would form membranes with a top hydrophilic layer of length A, a core hydrophobic layer of length B, and a bottom hydrophilic layer of length A.

Additionally, or alternatively, the polymer packing into the layer(s) of the membranes 20 may affect the hydrophilic ratio for each of the membranes 20, where the hydrophilic ratio may be defined as the ratio between molecular mass of the hydrophilic block and the total molecular weight (MW or M_{w}) of the block copolymer (BCP) (hydrophilic ratio=M_{w} hydrophilic block/M_{w} BCP). For examples: A-B diblock copolymer (FIG. 4A), hydrophilic ratio=x/(x+y/2); and A-B-A triblock copolymer (FIG. 4B), hydrophilic ratio=2x/(2x+y).

The present diblock and triblock copolymers may include any suitable combination of hydrophobic and hydrophilic blocks. In some examples, the hydrophilic A block may include a polymer selected from the group consisting of: N-vinyl pyrrolidone, polyacrylamide, zwitterionic polymer, hydrophilic polypeptide, nitrogen containing units, and poly(ethylene oxide) (PEO). Illustratively, the polyacrylamide may be selected from the group consisting of: poly(N-isopropyl acrylamide) (PNIPAM), and charged polyacrylamide, and phosphoric acid functionalized polyacrylamide. Examples of zwitterionic monomers that may be polymerized to form zwitterionic polymers include:

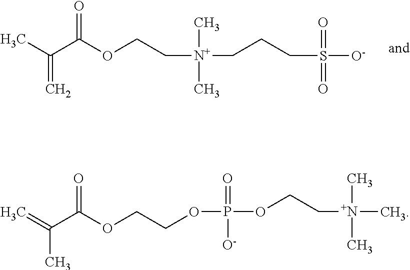 [SMILES: C=C(C)C(=O)OCC[N+](C)(C)CCCS(=O)(=O)[O-] ; C=C(C)C(=O)OCCOP(=O)([O-])OCC[N+](C)(C)C]

Examples of hydrophilic polypeptides include:

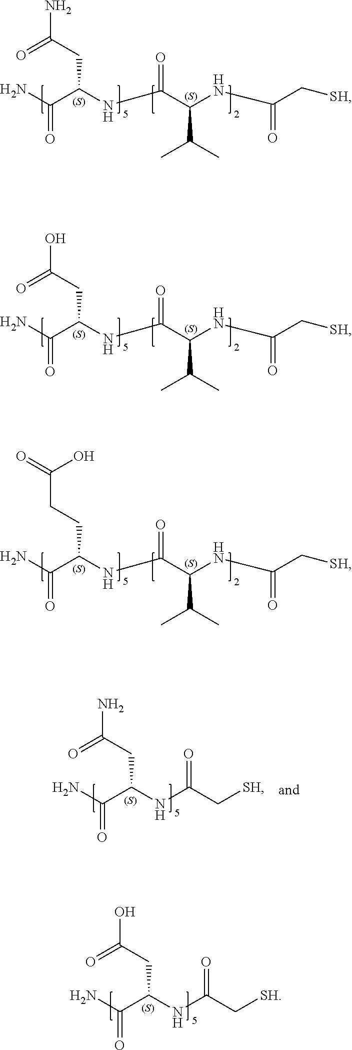

An example of a charged polyacrylamide is

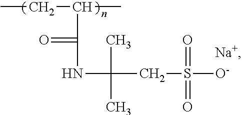

where n is between about 2 and about 100. Examples of nitrogen containing units include:

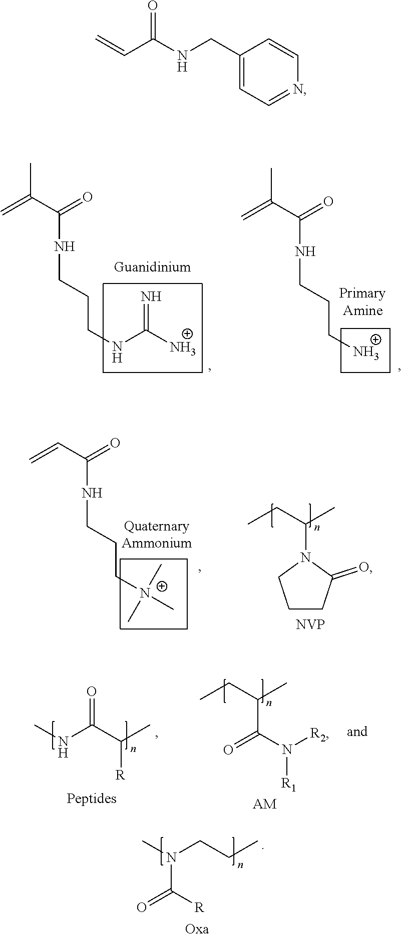

In some examples, the hydrophobic B block may include a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB). Examples of hydrogenated polydienes include saturated polybutadiene (PBu), saturated polyisoprene (PI), saturated poly(myrcene),

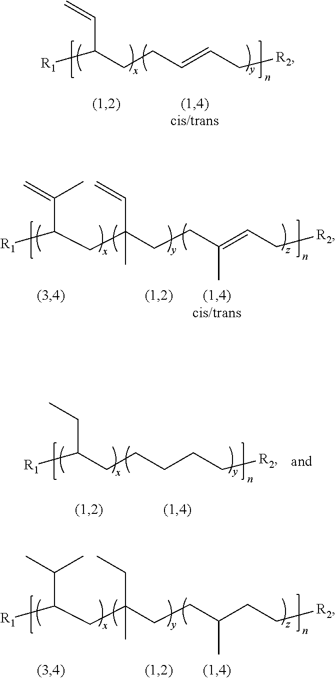

where n is between about 2 and about 100, x is between about 2 and about 100, y is between about 2 and about 100, z is between about 2 and about 100, R₁ is a functional group selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen, and R₂ is a reactive moiety selected from the group consisting of a maleimide group, an allyl group, a propargyl group, a bicyclononyne (BCN) group, a carboxylate group, an amine group, a thiol group, a dibenzocyclooctyne (DBCO) group, an azide group, an N-hydroxysuccinimide group, a biotin group, a carboxyl group, an N-hydroxysuccinimide (NHS)-activated ester, and other activated esters. In other examples of hydrogenated polydienes, R₁ is a reactive moiety selected from the group consisting of a maleimide group, an allyl group, a propargyl group, a BCN group, a carboxylate group, an amine group, a thiol group, a DBCO group, an azide group, an N-hydroxysuccinimide group, a biotin group, a carboxyl group, an NHS-activated ester, and other activated esters. An example of fluorinated polyethylene is

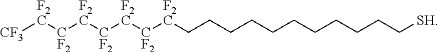 [SMILES: FC(F)(F)C(F)(F)C(F)(F)C(F)(F)C(F)(F)C(F)(F)C(F)(F)C(F)(F)CCCCCCCCCCCS]

Examples of hydrophobic polypeptides include:

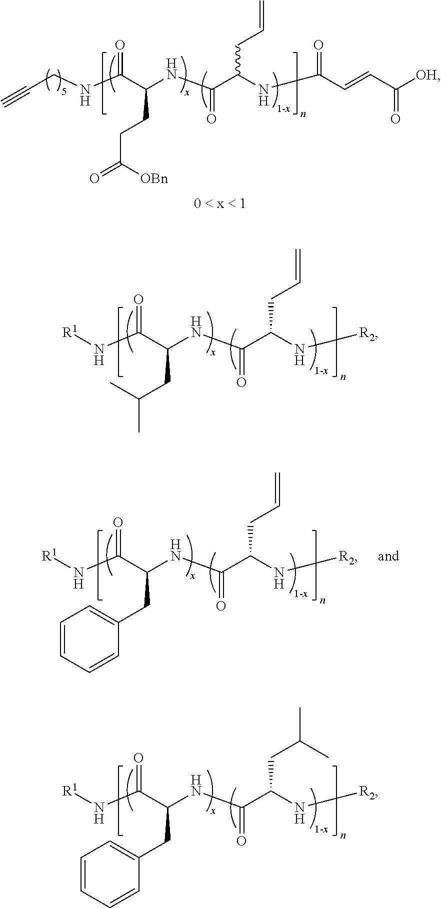

where n is between about 2 and about 100 and 0<x<1 in each structure.

In one example, an AB diblock copolymer includes PDMS-b-PEO, where “-b-” denotes that the polymer is a block copolymer. In another example, an AB diblock copolymer includes PBd-b-PEO. In still another example, an AB diblock copolymer includes PIB-b-PEO.

In one example, an ABA triblock copolymer includes PEO-b-PBd-b-PEO. In another example, an ABA triblock copolymer includes PEO-b-PDMS-b-PEO. In still another example, an ABA triblock copolymer includes PEO-b-PIB-b-PEO.

It will be appreciated that any suitable hydrophilic block(s) may be used with any suitable hydrophobic block(s). Additionally, in examples including two hydrophilic blocks, those blocks may be the same polymers as one another or may be different polymers from one another. Similarly, in examples including two hydrophobic blocks, those blocks may be the same polymers as one another or may be different polymers from one another.

The respective molecular weights, glass transition temperatures, and chemical structures of the hydrophobic and hydrophilic blocks suitably may be selected so as to provide the membrane 20 with appropriate stability for use and ability to insert the nanopore 22. For example, the respective molecular weights of the hydrophobic and hydrophilic blocks may affect how thick each of the blocks (and thus layers of the membrane 20) are, and may influence stability as well as capacity to insert the nanopore 22, e.g., through electroporation, pipette pump cycle, or detergent assisted pore insertion. Additionally, or alternatively, the ratio of molecular weights of the hydrophilic and hydrophobic blocks may affect self-assembly of those blocks into the layers of the membrane 20. Additionally, or alternatively, the respective glass transition temperatures (T_{g}) of the hydrophobic and hydrophilic blocks may affect the lateral fluidity of the layers of the membrane 20. As such, in some examples, it may be useful for the hydrophobic and/or hydrophilic blocks to have a T_{g} of less than the operating temperature of the device, e.g., less than room temperature, and in some examples less than about 0° C. Additionally, or alternatively, chemical structures of the hydrophobic and hydrophilic blocks may affect the way the chains get packed into the layers, and the stability of those layers.

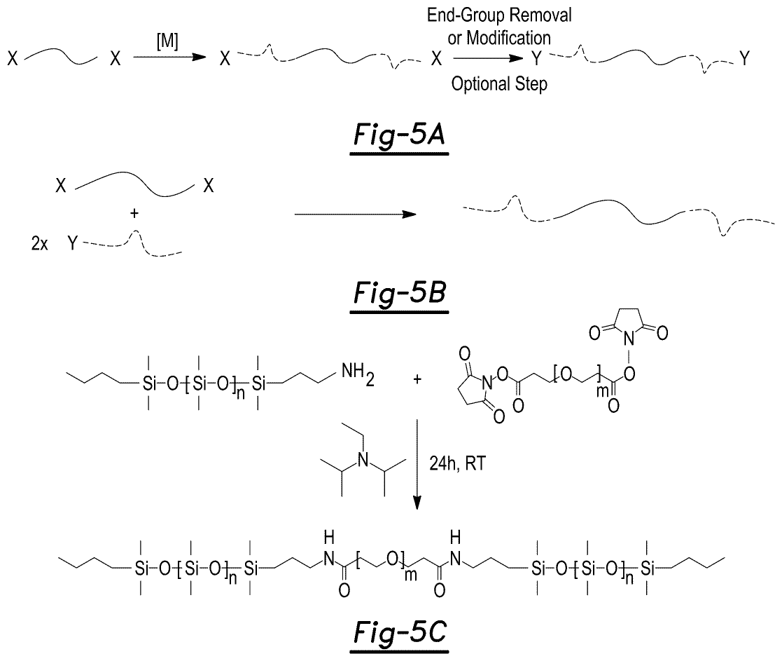

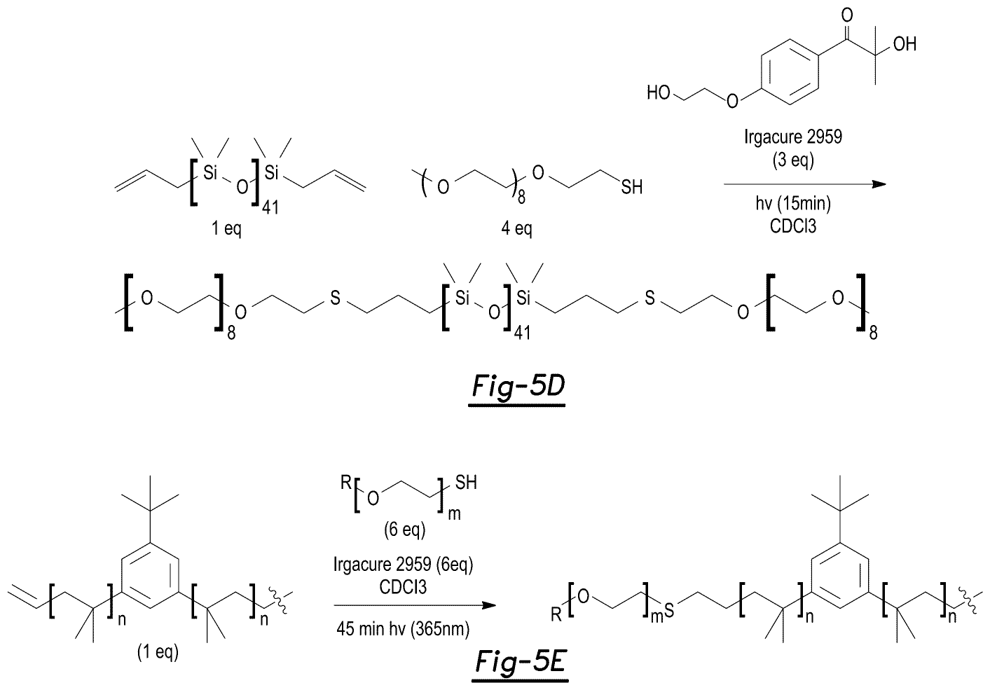

It will be appreciated that the present diblock and triblock copolymers may be made using any suitable combination of operations. FIG. 5A through FIG. 5D schematically illustrate example schemes for preparing block copolymers for use in the examples disclosed herein. In some examples, the present diblock and triblock copolymers may be made using a “macro-initiator” approach such as illustrated in FIG. 5A, in which one polymer block is made first and then used as an initiator (X) to grow one or more additional blocks using monomers [M]. Illustratively, operations for making a diblock copolymer may include polymerizing a plurality of hydrophilic monomers to form a hydrophilic polymer; forming an initiator at a terminal end of the hydrophilic polymer; and using the initiator to polymerize a plurality of hydrophobic monomers to form a hydrophobic polymer coupled to the hydrophilic polymer. Alternatively, operations for making a diblock copolymer may include polymerizing a plurality of hydrophobic monomers to form a hydrophobic polymer; forming an initiator at a terminal end of the hydrophobic polymer; and using the initiator to polymerize a plurality of hydrophilic monomers to form a hydrophilic polymer coupled to the hydrophobic polymer. Similarly, operations for making a triblock ABA copolymer may include polymerizing a plurality of hydrophobic monomers to form a hydrophobic polymer; forming initiators at respective terminal ends of the hydrophobic polymer; and using the initiators to polymerize a plurality of hydrophilic monomers to form a hydrophilic polymer coupled to each terminal end of the hydrophobic polymer. In such a “macro-initiator” approach, the initiator (X in FIG. 5A) suitably may be selected based on the particular monomers being used and the particular type of polymerization being performed. For example, for an atom transfer free radical polymerization (ATRP), the initiator may include bromine or chlorine. Or, for example, for a reversible addition fragmentation chain transfer (RAFT) polymerization, the initiator may include a chain transfer agent. After polymerization is complete, the end group(s) (X) may be modified or removed (e.g., to provide end group(s) Y in FIG. 5A).

In other examples, the present diblock and triblock copolymers may be made using a “coupling” approach, such as illustrated in FIG. 5B, in which polymer blocks are made separately and then coupled together using reactive moieties (X and Y). Illustratively, operations for making a diblock copolymer may include polymerizing a plurality of hydrophilic monomers to form a hydrophilic polymer; polymerizing a plurality of hydrophobic monomers to form a hydrophobic polymer; and coupling the hydrophilic polymer to the hydrophobic polymer. Operations for making a triblock copolymer may include polymerizing a plurality of hydrophilic monomers to form a hydrophilic polymer having terminal ends; polymerizing a plurality of hydrophobic monomers to form first and second hydrophobic polymers; and coupling the first and second hydrophobic polymers to respective terminal ends of the hydrophilic polymer. Alternatively, operations for making a triblock copolymer may include polymerizing a plurality of hydrophilic monomers to form first and second hydrophilic polymers; polymerizing a plurality of hydrophobic monomers to form a hydrophobic polymer having terminal ends; and coupling the first and second hydrophilic polymers to respective terminal ends of the hydrophobic polymer. In such a “coupling” approach, a terminal end of the hydrophobic polymer may include a first reactive moiety (Y in FIG. 5B), and a terminal end of the hydrophilic polymer may include a second reactive moiety (X in FIG. 5B) that reacts with the first reactive moiety to couple the hydrophilic polymer to the hydrophobic polymer. The reactive moieties (X and Y) suitably may be selected based on the particular polymers being coupled and the type of coupling being performed. For example, “Click” chemistry moieties may be used. Illustratively, one of the first and second reactive moieties may include an azide and the other of the first and second reactive moieties may include an alkyne; or one of the first and second reactive moieties may include a thiol and the other of the first and second reactive moieties may include an alkene; or one of the first and second reactive moieties may include a thiol and the other of the first and second reactive moieties comprises an alkyne. Or, for example, amide linkers may be formed. Illustratively, one of the first and second reactive moieties may include an amine and the other of the first and second reactive moieties may include N-hydroxysuccinimide (NHS). FIG. 5C illustrates an example in which the hydrophobic polymer is PDMS having an amine (NH₂) group at one of its terminal ends, the hydrophilic polymer is PEO having NHS at its terminal end, and the amine and NHS groups are reacted with one another in the presence of triisopropylamine to provide an ABA triblock copolymer. Another example of forming a triblock copolymer is shown in FIG. 5D. In this example, a PDMS-bis allyl is reacted by thiol-ene click chemistry with a PEG-thiol. For the reaction to proceed, it is carried out under an inert atmosphere using a degassed solvent (e.g., chloroform) and in the presence of a photoinitiator (e.g., IRGACURE® 2959), and under UV exposure (e.g., 365 nm UV light source) for 5-30 minutes.

Still another example of forming a triblock copolymer is shown in FIG. 5E. In this example, a PIB-bis allyl is reacted by thiol-ene click chemistry with a PEG-thiol. For the reaction to proceed, it is carried out under an inert atmosphere (dry Argon or dry Nitrogen) using a degassed solvent (e.g., chloroform) and in the presence of a photoinitiator (e.g., IRGACURE® 2959), and under UV exposure for 5-60 minutes. In some examples, the reaction is carried out under UV exposure for 2-180 minutes with a UV power ranging from 1 mW/cm² to 100 mW/cm². In some examples, the UV wavelength used is 365 nm.

For nanopore sequencing applications, membrane fluidity can be considered beneficial. Without wishing to be bound by any theory, the fluidity of a block copolymer membrane is believed to be largely imparted by the physical property of the hydrophobic “B” blocks. More specifically, B blocks including “low T_{g}” hydrophobic polymers (e.g., having a T_{g} below around 0° C.) may be used to generate membranes that are more fluid than those with B blocks including “high T_{g}” polymers (e.g., having a T_{g} above room temperature). For example, in certain examples, a hydrophobic B block of the copolymer has a T_{g} of less than about 20° C., less than about 0° C., or less than about −20° C.

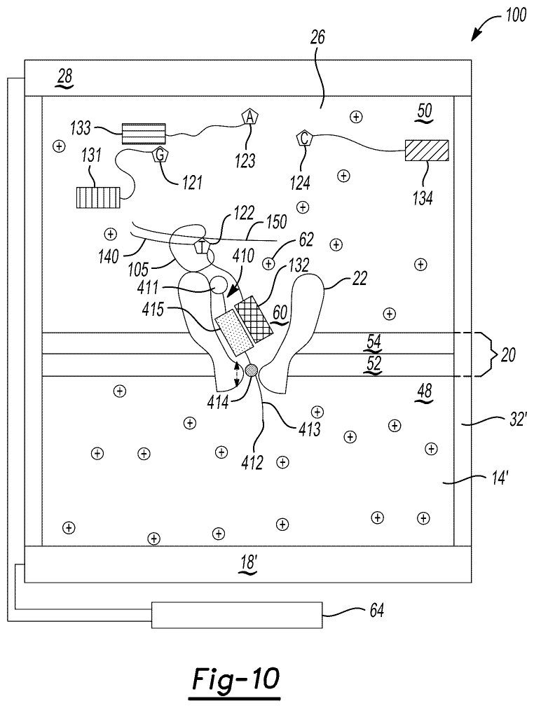

Hydrophobic B blocks with a low T_{g} may be used to help maintain membrane flexibility under conditions suitable for performing nanopore sequencing, e.g., in a manner such as described with reference to FIG. 10 through FIG. 14. In some examples, hydrophobic B blocks with a sufficiently low T_{g} for use in nanopore sequencing may include, or may consist essentially of, PIB, which may be expected to have a T_{g} in the range of about −75° C. to about −25° C. In other examples, hydrophobic B blocks with a sufficiently low T_{g} for use in nanopore sequencing may include, or may consist essentially of, PDMS, which may be expected to have a T_{g} in the range of about −135° C. (or lower) to about −115° C. In still other examples, hydrophobic B blocks with a sufficiently low T_{g} for use in nanopore sequencing may include, or may consist essentially of, PBd. Different forms of PBd may be used as B blocks in the present barriers. For example, the cis-1,4 form of PBd may be expected to have a T_{g} in the range of about −105° C. to about −85° C. Or, for example, the cis-1,2 form of PBd may be expected to have a T_{g} in the range of about −25° C. to about 0° C. Or, for example, the trans-1,4 form of PBd may be expected to have a T_{g} in the range of about −95° C. to about −5° C. In yet other examples, hydrophobic B blocks with a sufficiently low T_{g} for use in nanopore sequencing may include, or may consist essentially of, polymyrcene (PMyr), which may be expected to have a T_{g} in the range of about −75° C. to about −45° C. In yet other examples, hydrophobic B blocks with a sufficiently low T_{g} for use in nanopore sequencing may include, or may consist essentially of, polyisoprene (PIP). Different forms of PIP may be used as B blocks in the present barriers. For example, the cis-1,4 form of PIP may be expected to have a T_{g} in the range of about −85° C. to about −55° C. Or, for example, the trans-1,4 form of PIP may be expected to have a T_{g} in the range of about −75° C. to about −45° C.

Hydrophobic B blocks with a fully saturated carbon backbone, such as PIB, also may be expected to increase chemical stability of the block copolymer membrane. Additionally, or alternatively, branched structures within the hydrophobic B block, such as with PIB, may be expected to induce chain entanglement, which may be expected to enhance the stability of the block copolymer membrane. This may allow for a smaller hydrophobic block to be used, ameliorating the penalty of hydrophobic mismatch towards an inserted nanopore. Additionally, or alternatively, hydrophobic B blocks with relatively low polarity may be expected to be better electrical insulators, thus improving electrical performance of a device for nanopore sequencing (e.g., such as described with reference to FIG. 10 through FIG. 14).

In some examples of the AB copolymer shown below, including PBd as the B block and PEO as the A block, Ris a functional group selected from the group consisting of a carboxyl group (in acid or ion form), a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen; m=about 2 to about 100; and n=about 2 to about 100.

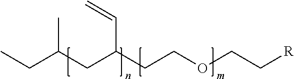

In some examples, R=OH; n=about 8 to about 50; and m=about 1 to about 20.
In other examples, R=OH; n=about 10 to about 15; and m=about 5 to about 15.

In some examples of the ABA copolymer shown below, including one or more PIB blocks as the B block and PEO as the A block, R₁ and R₂ are independently moieties selected from the group consisting of a carboxyl group (in acid or ion form), a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen; V is an optional group that corresponds to a bis-functional initiator from which the isobutylene may be propagated and can be tert-butylbenzene, a phenyl connected to the hydrophobic blocks via the para, meta, or ortho positions, naphthalene, another aromatic group, an alkane chain with between about 2 and about 20 carbons, or another aliphatic group; m=about 2 to about 100; and n=about 2 to about 100. V may be flanked by functional groups selected from the group consisting of a carboxyl group (in acid or ion form), a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen. When V is absent, only one PIB block is present and n=about 2 to about 100. L₁ and L₂ are independently linkers, which may include at least one moiety selected from the group consisting of an amide, a thioether (sulfide), a succinic group, a maleic group, a methylene, an ether, and a product of a click reaction.

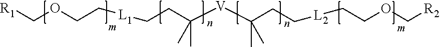

In some examples of the above structure, n=about 2 to about 50, and m=about 1 to about 50, R₁=R₂=COOH, V=tert-butylbenzene, and L₁=L₂=ethyl sulfide. In other examples, n=about 5 to about 20, m=about 2 to about 15, R₁=R₂=COOH, V=tert-butylbenzene, and L₁=L₂=ethyl sulfide. In still other examples, n=about 13 to about 19, m=about 2 to about 5, R₁=R₂=COOH, V=tert-butylbenzene, and L₁=L₂=ethyl sulfide. In yet other examples, n=about 7 to about 13, m=about 7 to about 13, R₁=R₂=COOH, V=tert-butylbenzene, and L₁=L₂=ethyl sulfide. In particular, in one example (the structure of which is shown below), n=16, m=3, R₁=R₂=COOH, V=tert-butylbenzene, and L₁=L₂=ethyl sulfide. In another example (the structure of which is shown below), n=10, m=10, and R₁=R₂=COOH, V=tert-butylbenzene, and L₁=L₂=ethyl sulfide. In another example (the structure of which is shown below), n=16, m=8, R₁=R₂=CH₃, V=tert-butylbenzene, and L₁=L₂=ethyl sulfide.

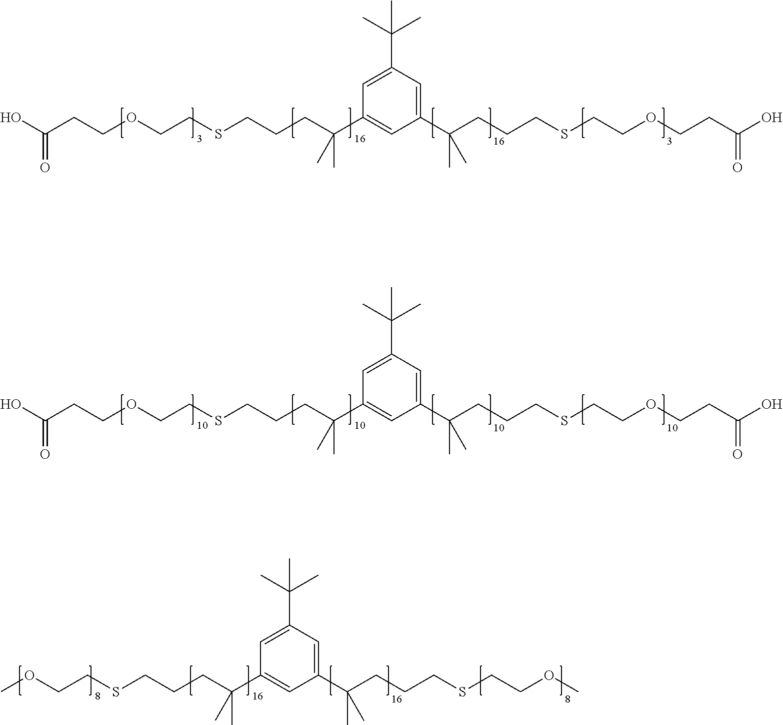

In some examples, multifunctional precursors may be sourced and used as precursors to the synthesis of bifunctional initiators to which V corresponds in the example further above. For example, the multifunctional precursor may be 5-tert-butylisophthalic acid (TBIPA) which can be synthesized into 1-(tert-butyl)-3,5-bis(2-methoxypropan-2-yl)benzene (TBDMPB) using reactions known in the art. In another example, TBIPA may be synthesized into 1-tert-butyl-3,5-bis(2-chloropropan-2-yl)benzene using reactions known in the art. The use of such bifunctional initiators allows cationic polymerization on both sides of the initiator, generating bifunctional PIBs, such as allyl-PIB-allyl, which can then be coupled to hydrophilic A blocks to generate ABA block copolymers including PIB as the B block. Here, although the bifunctional initiator may be located between first and second PIB polymers, it should be understood that the first and second PIB polymers and the bifunctional initiator (V) together may be considered to form a B block, e.g., of an ABA triblock copolymer.

In another example, an ABA triblock copolymer includes

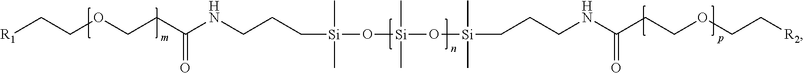

where m=about 2 to about 100, n=about 2 to about 100, p=about 2 to about 100, R₁ and R₂ are independently functional groups selected from the group consisting of a carboxyl group (in acid or ion form), a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen. In some examples, m=about 2 to about 30, n=about 25 to about 45, p=about 2 to about 30, R₁ and R₂ are independently functional groups selected from the group consisting of a carboxyl group (in acid or ion form), a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen. In some other examples, m=about 2 to about 15, n=about 30 to about 40, p=about 2 to about 15, R₁ and R₂ are independently functional groups selected from the group consisting of a carboxyl group (in acid or ion form), a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen. In some other examples, m=about 7 to about 11, n=about 35 to about 40, p=about 7 to about 11, R₁ and R₂ are independently functional groups selected from the group consisting of a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen. In further examples, m=about 2 to about 5, n=about 30 to about 37, p=about 2 to about 5, R₁ and R₂ are independently functional groups selected from the group consisting of a carboxyl group (in acid or ion form), a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen.

In particular, as shown below, in one example, m=3, n=34, p=3, and R₁=R₂=COOH. In another example shown below, m=9, n=37, p=9, and R₁=R₂=COOH.

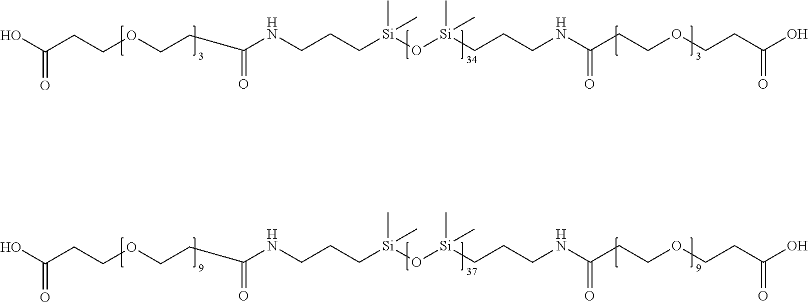

Still another example of the ABA copolymer includes

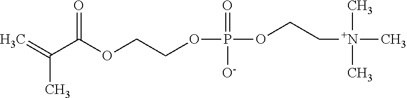 [SMILES: C=C(C)C(=O)OCCOP(=O)([O-])OCC[N+](C)(C)C]

as the A block, PDMS as the B block, and propanethiol as a linker.

In some examples of the AB copolymer shown below including a PIB block as the B block and PEO as the A block, R is a moiety selected from the group consisting of a carboxyl group (in acid or ion form), a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen; m=about 2 to about 100; n=about 2 to about 100; and L is a linker selected from the group consisting of an amide, a thioether (sulfide), a succinic group, a maleic group, a methylene, an ether, or a product of a click reaction.

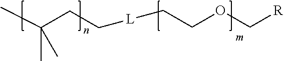

In particular, as shown below, in one example, n=13, m=8, R is methyl, and L is ethyl sulfide. In another example shown below, n=13, m=3, R is a carboxyl group, and L is ethyl sulfide. In still another example shown below, n=30, m=8, R is methyl, and L is ethyl sulfide. In yet another example shown below, n=30, m=3, R is a carboxyl group, and L is ethyl sulfide.

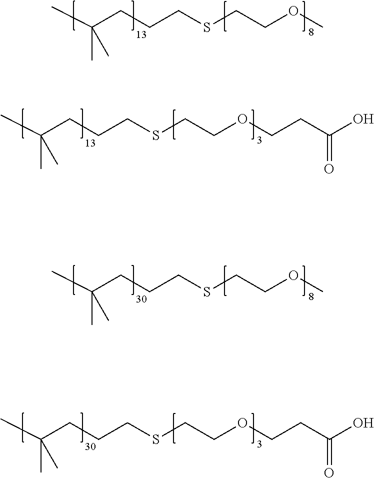

Other AB copolymers are shown below including a PIB block as the B block and PEO as the A block:

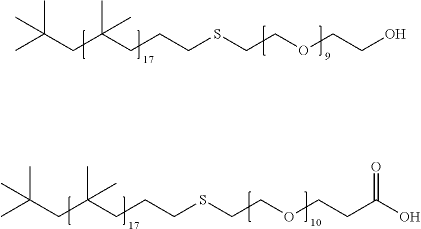

Still other AB copolymers are shown below including a PDMS block as the B block and PEO as the A block:

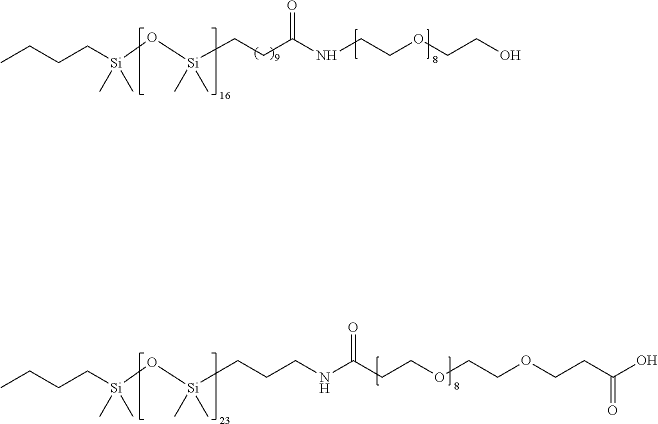

As shown in FIG. 1, FIG. 2, and FIG. 3, the fluidic device 10 and the nanopore instruments 100, 100′ further include nanopore(s) 22. The nanopore(s) 22 is/are attached to the membrane fragments 19 that are bound over the interstitial regions 16 during pre-operational processes, and is/are inserted within the suspended membrane(s) 20 that is/are operatively positioned across the aperture 24 during operational use. When in the operative position (as shown in FIG. 2), the nanopore 22 is disposed within the membrane 20 and provides an opening 60 into the well 14 or between the wells 14′ and 26. In the operative position, this opening 60 fluidically couples the trans well(s) 14′ to the cis well 26. As such, the opening 60 of nanopore 22 may provide a pathway for fluid 50 and/or fluid 48 to flow through the membrane 20. In the example of FIG. 2, a portion of salt 62 may move from the second side 46 of the membrane 20 to the first side 44 of the membrane 20 through the opening 60.

In the examples set forth herein, the nanopore(s) 22 is/are biological nanopore(s) or hybrid nanopore(s). In one example, the membrane fragments 19 and the membrane 20 formed therefrom are a polymeric membrane material (e.g., the diblock or triblock copolymers described herein); and the nanopore 22 is a biological nanopore or a hybrid nanopore. In a specific example, the polymeric membrane material (for 19 and 20) is a polybutadiene-polyethylene oxide block copolymer; and the nanopore 22 is Mycobacterium smegmatis porin A (MspA).

When in operational use, the nanopore instruments 100, 100′ include a first fluid 48 in the trans well 14′ and a second fluid 50 in the cis well 26. The first fluid 48 within trans well 14′ is in contact with the first side 44 of the membrane 20, and second fluid 50 within the cis well 26 is in contact with the second side 46 of the membrane 20.

The first fluid 48 may have a first composition including a first concentration of a salt 62, which salt may be represented for simplicity as positive ions (although it will be appreciated that counterions are also present). The second fluid 50 may have a second composition including a second concentration of the salt 62 that may be the same as, or different than, the first concentration. Any suitable salt or salts 62 may be used in first and second fluids 48, 50, e.g., ranging from common salts to ionic crystals, metal complexes, ionic liquids, or even water soluble organic ions. For example, the salt may include any suitable combination of cations (such as, but not limited to, H, Li, Na, K, NH₄, Ag, Ca, Ba, and/or Mg) with any suitable combination of anions (such as, but not limited to, OH, Cl, Br, I, NO₃, ClO₄, F, SO₄, and/or CO₃²⁻ . . . ). In one example, the salt includes potassium chloride (KCl). It will also be appreciated that the first and second fluids optionally may include any suitable combination of other solutes. Illustratively, first and second fluids 48, 50 may include an aqueous buffer (such as N-(2-hydroxyethyl) piperazine-N′-2-ethanesulfonic acid (HEPES), commercially available from Fisher BioReagents). Any difference between the first and second concentrations of salt 62 and/or between other components of the first and second fluids 48, 50, may generate osmotic pressure 66 across the membrane 20. As provided herein, the diblock or triblock copolymer used in membrane 20 may be selected so as to provide the membrane 20 with sufficient stability for use over a desired period of time, e.g., for use over the course of sequencing a polynucleotide.

During pre-operational processes, such as handling, shipping and/or storage, the wells 14′, 26 may or may not contain fluid. In one example, the trans well(s) 18′ and the cis well 26 contain a buffer, an example of which is an electrode buffer. Any of the fluids described herein may be used for shipping and/or storage as long as they do not interfere with the components of the device 10/instrument 100, 100. The fluid may be replaced or refreshed just prior to operational use. As specific examples, the buffer for shipping and/or storage is KCl or HEPES, alone or in combination with a reducing agent, such as TCEP ((tris(2-carboxyethyl)phosphine)). In another example, the trans well(s) 18′ and the cis well 26 are free of a liquid during shipping and/or storage.

### Methods for Making the Fluidic Device and Nanopore Instruments

FIG. 6A through FIG. 6C illustrate the formation of the fluidic device 10 so that the membrane fragments 19 and nanopores 22 are in the inoperable position (e.g., for handling, storage, and/or shipping), and FIG. 7A through FIG. 7C illustrate the painting of the membrane 20 (using the membrane fragments 19) and nanopore 22 into the operable position (e.g., for sequencing). While the methods are shown and described in reference to the fluidic device 10, it is to be understood that the method may be used with either of the nanopore instruments 100, 100′ disclosed herein.

The method shown in FIG. 6A through FIG. 6C includes forming a membrane 20 across an aperture 14 of a well 14, 14′ defined by a support structure 12, 12′, 12″ wherein the membrane 20 is supported by interstitial regions 16 of the support structure 12, 12′, 12″; inserting a nanopore 22 into the membrane 20; and breaking the membrane 20 to form membrane fragments 19 that become bound over the interstitial regions 16, whereby the nanopore 22 or nanopore subunits non-specifically binds/bind over the interstitial regions 16.

As shown in FIG. 6A, the membrane 20 is initially formed over the aperture 24 of the well 14, 14′. The membrane 20 may be any of the examples set forth herein.

The membrane material may be mixed with a hydrophobic (non-polar) solvent, such as octane, to form a membrane mixture. The membrane material may be painted across the aperture 24. Suitable techniques for painting the membrane 20 include brush painting (manual), mechanical painting (e.g., using stirring bar), bubble painting, or water-oil-water painting. With bubble painting, an aqueous solution is flowed through the channel (e.g., cis well 26, not shown in FIG. 6A), followed by an air (or other gas) gap, followed by the membrane mixture, and then another air (or other gas) gap. This process creates a bubble with the membrane mixture at its exterior. Additional aqueous solution is introduced into the channel to push the bubble and membrane mixture through the channel. As the bubble passes over the aperture, the membrane 20 is formed. With water-oil-water painting, an aqueous solution is first flowed through the channel (e.g., cis well 26), followed by the membrane mixture, followed by additional aqueous solution. The second aqueous solution pushes the membrane material across the aperture 24. These techniques result in the formation of the membrane 20, as shown in FIG. 6A. The membrane 20 is supported by the support structure 12, 12′, 12″ and is suspended across the aperture 24. In some examples, the membrane 20 has a painting yield of 90% or more, or 95% or more.

As shown in FIG. 6A, an annulus 68 including the hydrophobic (non-polar) solvent, and which also may include other compound(s), may adhere to support structure 12 (e.g., at patterned layer 32). The annulus 68 may also support a portion of the membrane 20, e.g., may be located within the membrane 20 (here, between layer 52 and layer 54).

The nanopore 22 is then inserted into the membrane 20, as shown in FIG. 6B. Suitable techniques for inserting a nanopore 22 into a suspended membrane 20 include electroporation, pipette pump cycle, and detergent assisted pore insertion. In one example, a solution of nanopores 22 in a buffer is added to the well 14 (or a channel overlying the well 14) or wells 14′, 26, and the pores 22 are respectively inserted into individual membranes 20 using electrical pulses.

In some examples, the membrane 20 has a pore insertion voltage between about 300 mV and about 900 mV. In some examples, the membrane 20 has a single pore percentage after insertion of 85% or more, 90% or more, or 95% or more. In some examples, the membrane 20 has a single pore survival rate of 90% or more, or 95% or more. In some examples, the membrane 20 has a single pore current standard deviation of 2 pA or less, 1 pA or less, or 0.5 pA or less.

In some examples, a waveform is applied to the membrane 20. The waveform is made of a train of positive voltage micro pulses, spaced by negative voltage periods at −100 mV for 100 ms. The train of positive voltage pulses has a total of 20 pulses, with duration of 10 μs. The spacings between them have a set duration value of 30 ms and a voltage held at +50 mV. During a first cycle, the waveform may be applied continuously for a period of 5 minutes and the magnitude of the pulses kept at +700 mV. In further applied cycles (applied again for 5 mins each), the pulsing intensity is increased from +700 mV to +1200 mV in 100 mV steps, for a total of six cycles. In some examples, the membrane survival rate under such a waveform is 60% or more, 80% or more, 90% or more, or 95% or more. In some examples, the voltage at 50% membrane survival is 1000 mV or more, or 1200 mV or pore. In some examples, the voltage at 50% membrane and single pore survival is 900 mV or more, or 1000 mV or more.

Additionally, the membrane 20 has an open pore current at 100 mV of 95 pA or more, or 100 pA or more. In some examples, the membrane 20 has an open pore current at 50 mV of 32 pA or more, 34 pA or more, or 36 pA or more. In some examples, the barrier-pore RMS noise is 2.2 pA or less, 2.0 pA or less, 1.8 pA or less, 1.6 pA or less, or 1.5 pA or less. In some examples, the membrane 20 has a signal-to-noise ratio of 40 or more, 50 or more, 60 or more, or 70 or more.

After initial pore 22 insertion (FIG. 6B), the excess of the nanopore solution is washed away with buffer.

Tools for forming suspended membranes 20 using synthetic polymers, and for inserting nanopores 22 in the suspended membranes 20 are commercially available, such as the Orbit 16 TC platform available from Nanion Technologies Inc. (California, USA).

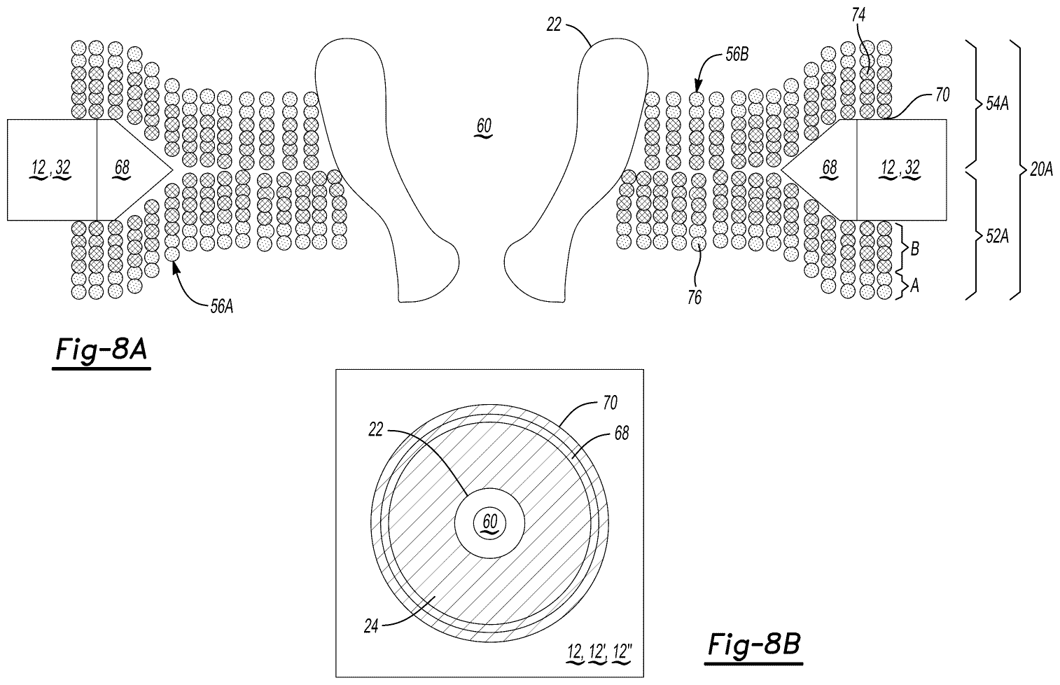

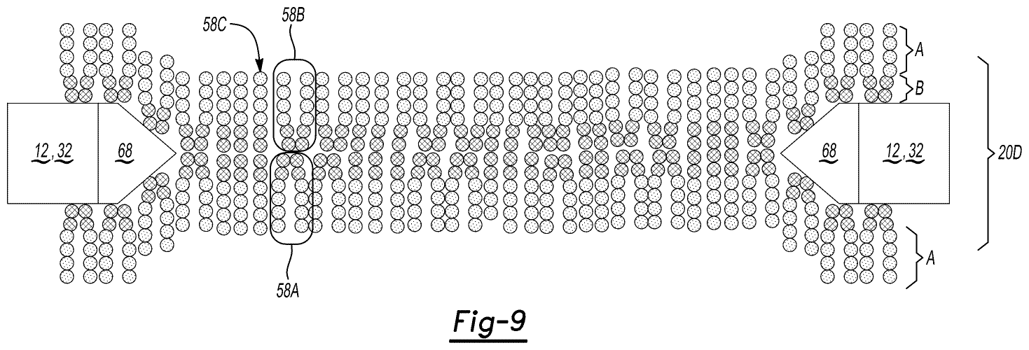

FIG. 8A and FIG. 8B schematically illustrate plan and cross-sectional views of further details of one example of the membrane 20 and nanopore 22. More specifically, in the example illustrated in FIG. 8A and FIG. 8B, membrane 20A may be suspended using the support structure 12 (e.g., at patterned layer 32) defining the aperture 24. For example, support structure 12 may include a patterned layer 32 having an aperture 24 defined therethrough, e.g., a substantially circular aperture, or an aperture having another shape. Additionally, or alternatively, the patterned layer 32 may include one or more features, such as a lip or ledge (e.g., interstitial regions 16), on either side of the well 14, 14′. Examples of materials which may be included in the support structure 12 are provided further above.

As shown in detail in FIG. 8A, the annulus 68 may adhere to support structure 12 and may support a portion of membrane 20, e.g., may be located within membrane 20 (here, between layer 52A and layer 54A). Additionally, annulus 68 may taper inwards in a manner such as illustrated in FIG. 8A.

An outer portion of the molecules 56B, e.g., those defining the layer 54A, may be disposed on the support structure 12 (e.g., the portion extending between aperture 24 and membrane periphery 70, see FIG. 8B), while an inner portion of the molecules 56A, e.g., those defining layer 52A, may form a freestanding portion of the membrane 20 (e.g., the portion within aperture 24, a part of which is supported by annulus 68). The membrane 20 may be prepared, and the nanopore 22 may be inserted into the freestanding portion of the membrane 20, using operations described herein. Although FIG. 8A and FIG. 8B illustrate the nanopore 22 within the membrane 20, it should be understood that the nanopore 22 may be omitted, and that the membrane 20 may be used for any suitable purpose. More generally, it should be appreciated that while the membranes 20 described herein are particularly suitable for use with nanopores 22 (e.g., for nanopore sequencing such as described with reference to FIG. 10 through FIG. 14), the present membranes 20 need not necessarily have nanopores 22 inserted therein.

In the example illustrated in FIG. 8A, the membrane 20A may include first layer 52A including a first plurality of amphiphilic molecules 56A and second layer 54A including a second plurality of the amphiphilic molecules 56B contacting the first plurality of amphiphilic molecules 56A. In the example illustrated in FIG. 8A, the copolymer is a diblock copolymer (AB) such as described with reference to FIG. 4A. Here, each molecule 56A, 56B includes a hydrophobic “B” block (within which circles 74 with darker fill represent hydrophobic monomers) and a hydrophilic “A” block (within which circles 76 with lighter fill represent hydrophilic monomers) coupled directly or indirectly together. In other examples, such as will be described with reference to FIG. 9, the copolymer instead may include a triblock copolymer (e.g., ABA). In the example illustrated in FIG. 8A, the hydrophilic blocks A of the first plurality of molecules 56A may form a first outer surface of the membrane 20, e.g., the surface 44 contacting fluid 48 in the well 14, 14′. The hydrophilic blocks A of the second plurality of molecules 56B may form a second outer surface of the membrane 20, e.g., the surface 46 of contacting fluid 50 in the well 26. The hydrophobic blocks B of the first and second pluralities of molecules 56A, 56B may contact one another within the barrier.

Although FIG. 8A and FIG. 8B illustrate a suspended membrane 20A that includes a diblock copolymer, it will be appreciated that suspended membranes 20 that include other types of polymers provided herein, are similarly contemplated. FIG. 9 schematically illustrates an alternative barrier that may be used in the example described with reference to FIG. 8A and FIG. 8B. FIG. 9 illustrates a hybrid 20D of the membranes 20B, 20C, which is suspended using the support structure 12, and the annulus 68 as described with reference to FIG. 8A and FIG. 8B. In this example, the membrane 20D includes molecules of an ABA triblock copolymer such as described with reference to FIG. 4B and FIG. 4C. Here, the triblock copolymer includes hydrophobic “B” sections coupled to and between hydrophilic “A” sections. In the example shown in FIG. 9, each individual ABA molecule may be in one of two arrangements. For example, ABA molecules 58C may extend through the layer in a linear fashion, with an “A” section on each side of the barrier and the “B” section in the middle of the barrier. Or, for example, ABA molecules 58A, 58B may extend to the middle of the membrane 20D and then fold back on themselves, so that both “A” sections are on the same side of the membrane 20D and the “B” section is in the middle of the membrane 20D. Accordingly, in this example, membrane 20D may be considered to be partially a single layer and partially a bilayer. In other examples (as shown and described in reference to FIG. 4C), the membrane 20C substantially includes molecules 58C which extend through the layer in linear fashion. This membrane 20C may substantially be a monolayer. In still other examples (as shown and described in reference to FIG. 4B), the membrane 20B substantially includes molecules 58A and 58B which extend to approximately the middle of the layer and then fold back on themselves. This membrane 20B may substantially be a bilayer. A nanopore, not specifically shown in FIG. 9, may be inserted into any of membranes 20B through 20D in a manner similar to that described elsewhere herein, e.g., as illustrated in FIG. 8A and FIG. 8B.

In other examples, the nanopores 22 are not inserted into the membranes 20. Rather, the nanopores 22 may be introduced and allowed to incubate and non-specifically bind to the membranes 20. The membrane 20 may then be broken as described herein.

Referring now to FIG. 6C, after the membrane 20 is formed and the nanopore 22 is inserted therein, the membrane-pore constructs are broken.

The membranes 20 can be broken by exposure to high electrical pulses, a surfactant that disrupts the membrane 20, osmostic pressure, or high velocity buffer flow.

The membranes 20 may be exposed to high electrical pulses to break the membrane 20. In other words, the membranes 20 are zapped to form the membrane fragments 19. The membrane 20 may have a survival rate of 70% or more, 80% or more, 90% or more, or 95% or more when subjected to a voltage of 450 mV across the membrane 20. As described herein, the membrane 20 has a pore insertion voltage between about 300 mV and about 900 mV. As such, to zap the membrane 20 and break the membrane-pore constructs shown in FIG. 6B, voltages ranging from 1V to 2V are applied.

The membranes 20 may be exposed to a surfactant to break the membrane 20. In an example, the surfactant is a non-ionic surfactant, such as octylpolyoxyethylene (oPOE).

The membranes 20 may be exposed to osmostic pressure to break the membrane 20. The osmotic pressure needed to break the membrane 20 will depend, in part, upon the volume of the well 14, 14′ and channel (e.g., cis well 26) and the amount of membrane material that is present. In one example, a difference in salt concentration between the well 14, 14′ and the channel (e.g., cis well 26) may create a suitable pressure to break the membrane 20. In one example, 400 mM KCl in the trans well 14′ and water in the cis well 26 creates suitable membrane-breaking pressure.

The membranes 20 may be exposed to high velocity buffer flow to break the membrane 20. As one example, a flow of 1 mL/s through the channel may be sufficient to break the membrane 20.

After the membranes 20 are broken, the fluidic device 10 or nanopore instruments 100, 100′ may be allowed to incubate for a time. During incubation, the membrane fragments 19, and any nanopore 22 retained therein, may retract to the interstitial regions 16. Hydrophobic interaction may help to bind the membrane fragments 19 to the interstitial regions 16. For example, the amphiphilic block copolymer in oil is capable of adhering to the hydrophobic surface of the support structure 32, 32′, 32″.

The hydrophobic solvent may be present in the device 10/instrument 100, 100′ during incubation.

After membrane 20 breakage and membrane fragment 19 and nanopore 22 repositioning, the fluidic device 10 or nanopore instrument 100, 100′ may be washed with a buffer to remove any loose membrane material.

The formation of the membrane fragments 19 prepares the fluidic device 10 or nanopore instrument 100, 100′ for pre-operational processes, such as handling, shipping, and/or storage. In this form, the fluidic device 10 or nanopore instrument 100, 100′ includes all of the materials for preparing the device 10 or instrument 100, 100′ for operational use, but does not include the membrane 20 or the nanopore 22 in an operative position. Thus, the membrane fragments 19 are not suspended across the aperture 24, and thus are not susceptible to breakage during handling, shipping, and/or storage. In one example, the fluidic device 10 or nanopore instrument 100, 100′ can be stored at room temperature for at least 2 months.

In some instances, less than 100% of the membranes 20 are broken after the exposure to the high electrical pulse, the surfactant, etc. These membranes 20 may or may not withstand storage and/or shipping. In these examples, the painting process described in FIG. 7B may be performed in the presence of additional membrane material in order to generate a fresh membrane 20 or to repaint the potentially weakened membrane 20 that remains across the aperture 24. Additionally, in some instances, the nanopore 22 may be ejected from the intact membrane 20 during shipping and/or storage. In some of these instances, the ejected nanopore 22 may non-specifically bind to the membrane 20 or the membrane fragments 19 present in the device 10/instrument 100, 100′. This nanopore can be reinserted during the method described in reference to FIG. 7A through FIG. 7C. In some other of these instances, the ejected nanopore 22 may break into its nanopore subunits, and the subunits may bind over the membrane 20 or the membrane fragments 19 present in the device 10/instrument 100, 100′. The nanopore subunits can be reassembled during repainting, when a fresh buffer is introduced into the dry device 10/instrument 100, 100′, or when exposed to other suitable conditions that enable self-assembly. The subunits that self-assemble may or may not be from the same original nanopore 22, but those that self-assemble are of the same nanopore type.

In other instances, the membranes 20 are not broken after the exposure to the high electrical pulse, the surfactant, etc. These membranes 20 may or may not withstand storage and/or shipping. In these examples, the painting process described in FIG. 7B may be performed in the presence of additional membrane material in order to generate a fresh membrane 20 or to repaint the potentially weakened membrane 20 that remains across the aperture 24. Additionally, in some instances, the nanopore 22 may be ejected from the intact membrane 20 during shipping and/or storage. In some of these instances, the ejected nanopore 22 may non-specifically bind to the membrane 20 or the membrane fragments 19 present in the device 10/instrument 100, 100′. This nanopore can be reinserted during the method described in reference to FIG. 7A through FIG. 7C. In some other of these instances, the ejected nanopore 22 may break into its nanopore subunits, and the subunits may bind over the membrane 20 or the membrane fragments 19 present in the device 10/instrument 100, 100′. The nanopore subunits can be reassembled during repainting, when a fresh buffer is introduced into the dry device 10/instrument 100, 100′, or when exposed to other suitable conditions that enable self-assembly. The subunits that self-assemble may or may not be from the same original nanopore 22, but those that self-assemble are of the same nanopore type.

The fluidic device 10 or nanopore instrument 100, 100′ shown in FIG. 6C and FIG. 7A (i.e., with the membrane fragments 19 and the nanopore 22 in the inoperative position) is pre-treated with the materials for subsequent membrane regeneration and nanopore insertion. This is shown in FIG. 7A through FIG. 7C.

The method shown in FIG. 7A through FIG. 7C includes introducing a gas bubble into a flow through channel of a fluidic device 10 including: a support structure 12, 12′, 12″ defining a well 14, 14′ and interstitial regions 16 surrounding the well 14, 14′, the flow through channel (e.g., cis well 26) being in fluid communication with the well 14, 14′ and the interstitial regions 16; an electrode 18, 18′ operatively positioned at a bottom of the well 14, 14′; membrane fragments 19 bound over the interstitial regions 16; and a nanopore 22, nanopore subunits, or a combination thereof bound over the interstitial regions 16, wherein introducing the gas bubble causes the membrane fragments 19 to form a membrane 20 across an aperture 24 leading to the well 14; and applying a voltage to the electrode 18, 18′, thereby causing insertion of the nanopore 22 or a reassembled nanopore into the formed membrane 20.

At the point of use, the membrane fragments 19 (of the device 10 or instrument 100, 100′ depicted in FIG. 7A) can be regenerated by passing a gas (e.g., air) bubble or a series of gas bubbles in a buffer through a flow channel overlying the fluidic device 10 or through the cis well 26 of the nanopore instrument 100, 100′. The air bubble flushes through the channel, allowing repainting of the membranes 20. There is a correlation between bubble size and channel dimension, and the volume of membrane fragments 19 is sufficient to pass over all the wells 14, 14′ to be repainted. In an example, the air bubble is several-microliters and is capable of moving the membrane fragments 19 that overlie the interstitial regions 16. This forms the membrane 20 across the aperture 24. In some instances, additional membrane material may be added to aid in regenerating the membranes 20.

Next, the nanopore 22 can be reinserted using electrical pulses, examples of which are described in reference to FIG. 6B. The membrane 20 and nanopore 22 are then in an operative position, as shown in FIG. 7C, and the device 10 or nanopore instrument 100, 100′ can be used in a sequencing operation. When nanopore subunits are present in the device 10/instrument 100, 100′, the nanopore subunits can be reassembled to form the nanopore 22, and then the nanopore 22 can be inserted into the membrane 20.

When the method described in reference to FIG. 7A through FIG. 7C is performed using the nanopore instrument 100′, the well 14, 14′ is one of a plurality of trans wells 14′; the interstitial regions 16 surround each of the plurality of trans wells 14′; the flow through channel is in fluid communication with each of the plurality of trans wells (i.e., the flow through channel is the cis well 26); and introducing the gas bubble into the flow through channel involves introducing the gas bubble into an inlet 36 and guiding the gas bubble from the inlet 36 through the flow through channel in a single direction to an outlet 38, thereby causing the membrane fragments 19 to form the membrane 20 across respective apertures 24 of at least some of the plurality of trans wells 14′.

Also when the method described in reference to FIG. 7A through FIG. 7C is performed using the nanopore instrument 100′, respective electrodes 18′ are positioned at the bottom of each of the plurality of trans wells 14′; and voltage pulses are applied to each of the respective electrodes 18′, thereby inserting a nanopore 22 or a reassembled nanopore into at least some of the repainted membranes 20.

The steps of the method shown in FIG. 7A and FIG. 7B may be repeated (i.e., introducing multiple gas bubbles in series) if the desired number of membranes 20 are not repainted after the first pass. Repeating these steps may be performed as long as a sufficient amount of membrane material remains in the device 10/instrument 100, 100′ for the repainting process. Once the desired number of membranes 20 are repainted, electrical pulsing can be performed until a sufficient number of nanopores 22 are inserted (FIG. 7C).

In another example method, the membrane fragments 19 can be formed into the membrane 20 using the water-oil-water painting method described herein or using a modification of this painting method where gas-oil-gas is used instead.

### Nanopore Sequencing Methods

FIG. 10 through FIG. 14 illustrate different operational uses of the sequencing instrument 100. It is to be understood that the entire support structure 12, 12′, 12″, including substrate 30 and interstitial regions 16 are not shown.

It will further be appreciated that the present membranes 20 may be used in any suitable device or application. For example, FIG. 10 schematically illustrates a cross-sectional view of an example use of the instrument 100 of FIG. 2. The instrument 100 illustrated in FIG. 10 may be configured to include the cis well 26, membrane 20 (which may have a configuration such as described with reference to FIGS. 4A-4C, 8A-8B, and 9 (that is, membrane 20 may be suspended using the support structure 12, and may include any AB or ABA copolymer provided herein), first and second fluids 48, 50, and nanopore 22 in a manner described herein. In the example illustrated in FIG. 10, the fluid 50 may include a plurality of each of nucleotides 121, 122, 123, 124, e.g., G, T, A, and C, respectively. Each of the nucleotides 121, 122, 123, 124 in the fluid 50 may be coupled to a respective label 131, 132, 133, 134, which is coupled to the nucleotide 121, 122, 123, 124 via an elongated body (elongated body not specifically labeled). The fluid 50 may also include or have introduced thereto a polymerase 105. Alternatively, the polymerase 105 may be coupled to the nanopore 22 or the membrane 20, e.g., via a suitable elongated body (not specifically illustrated). The instrument 100 may further include first and second polynucleotides 140, 150 in a manner such as illustrated in FIG. 10. Polymerase 105 may be included for sequentially adding nucleotides of the plurality to the first polynucleotide 140 using a sequence of the second polynucleotide 150 as a template strand. For example, at the particular time illustrated in FIG. 10, polymerase 105 incorporates nucleotide 122 (T) into first polynucleotide 140, which is hybridized to second polynucleotide 150 to form a duplex. At other times (not specifically illustrated), polymerase 105 sequentially may incorporate other of nucleotides 121, 122, 123, 124 into first polynucleotide 140 using the sequence of second polynucleotide 150 as the template strand.

The circuitry 64 illustrated in FIG. 10 may be configured to detect changes in an electrical characteristic of the opening 60 responsive to the polymerase 105 sequentially adding nucleotides 121, 122, 123, 124 of the plurality to the first polynucleotide 140 using a sequence of the second polynucleotide 150. In the example illustrated in FIG. 10, the nanopore 22 may be coupled to permanent tether 410 which may include head region 411, tail region 412, elongated body 413, reporter region 414 (e.g., an abasic nucleotide), and moiety 415. Head region 411 of tether 410 is coupled to nanopore 22 via any suitable chemical bond, protein-protein interaction, or any other suitable attachment that is normally irreversible. Head region 411 can be attached to any suitable portion of nanopore 22 that places reporter region 414 within opening 60 and places moiety 415 sufficiently close to polymerase 105 so as to interact with respective labels 131, 132, 133, 134 of nucleotides 121, 122, 123, 124 that are acted upon by polymerase 105. Moiety 415 respectively may interact with labels 131, 132, 133, 134 in such a manner as to move reporter region 414 within opening 60 and thus alter the rate at which salt 62 moves through opening 60, and thus may detectably alter the electrical conductivity of opening 60 in such a manner as to be detected by circuitry 64. For further details regarding use of permanent tethers coupled to nanopores to sequence polynucleotides, see U.S. Pat. No. 9,708,655, the entire contents of which are incorporated by reference herein.

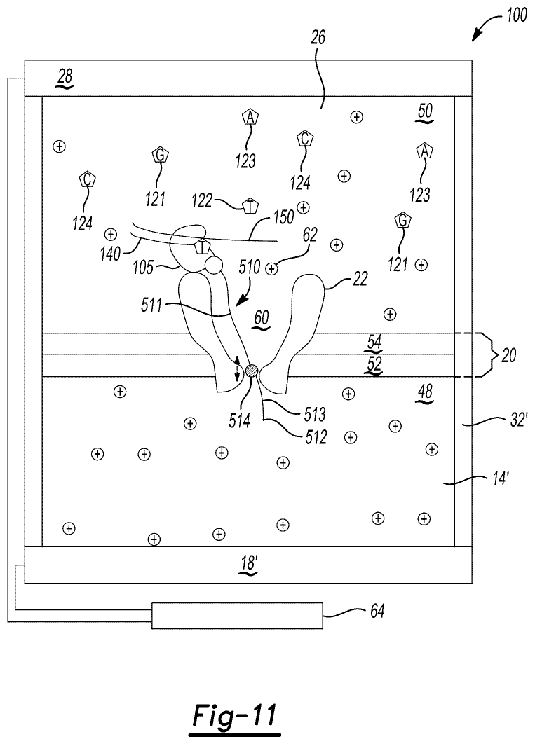

FIG. 11 schematically illustrates a cross-sectional view of another example use of the instrument of FIG. 2. As illustrated in FIG. 11, instrument 100 may include cis well 26, membrane 20 (which may have a configuration such as described with reference to FIG. 4A-4C, 8A-8B, or 9), first and second fluids 48, 50, nanopore 22, and first and second polynucleotides 140, 150, all of which may be configured similarly as described with reference to FIG. 10. In the example illustrated in FIG. 11, nucleotides 121, 122, 123, 124 need not necessarily be coupled to respective labels. Polymerase 105 may be coupled to nanopore 22 and may be coupled to permanent tether 510 which may include head region 511, tail region 512, elongated body 513, and reporter region 514 (e.g., an abasic nucleotide). Head region 511 of tether 510 is coupled to polymerase 105 via any suitable chemical bond, protein-protein interaction, or any other suitable attachment that is normally irreversible. Head region 511 can be attached to any suitable portion of polymerase 105 that places reporter region 514 within opening 60. As polymerase 105 interacts with nucleotides 121, 122, 123, 124, such interactions may cause polymerase 105 to undergo conformational changes. Such conformational changes may move reporter region 514 within opening 60 and thus alter the rate at which salt 62 moves through opening 60, and thus may detectably alter the electrical conductivity of opening 60 in such a manner as to be detected by circuitry 64. For further details regarding use of permanent tethers coupled to polymerases to sequence polynucleotides, see U.S. Pat. No. 9,708,655, the entire contents of which are incorporated by reference herein.

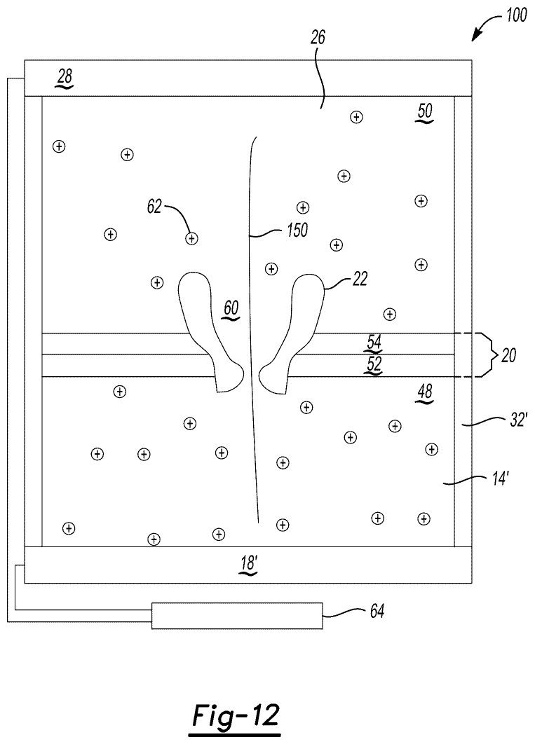

FIG. 12 schematically illustrates a cross-sectional view of another example use of the instrument of FIG. 2. As illustrated in FIG. 12, instrument 100 may include cis well 26, membrane 20 (which may have a configuration such as described with reference to FIG. 4A-4C, 8A-8B, or 9), first and second fluids 48, 50, and nanopore 22, all of which may be configured similarly as described with reference to FIG. 10. In the example illustrated in FIG. 12, polynucleotide 150 is translocated through nanopore 22 under an applied force, e.g., a bias voltage that circuitry 64 applies between electrode 18′ and electrode 28. As bases in polynucleotide 150 pass through nanopore 22, such bases may alter the rate at which salt 62 moves through opening 60, and thus may detectably alter the electrical conductivity of opening 60 in such a manner as to be detected by circuitry 64. For further details regarding use of nanopores to sequence polynucleotides being translocated therethrough, see U.S. Pat. No. 5,795,782, the entire contents of which are incorporated by reference herein.

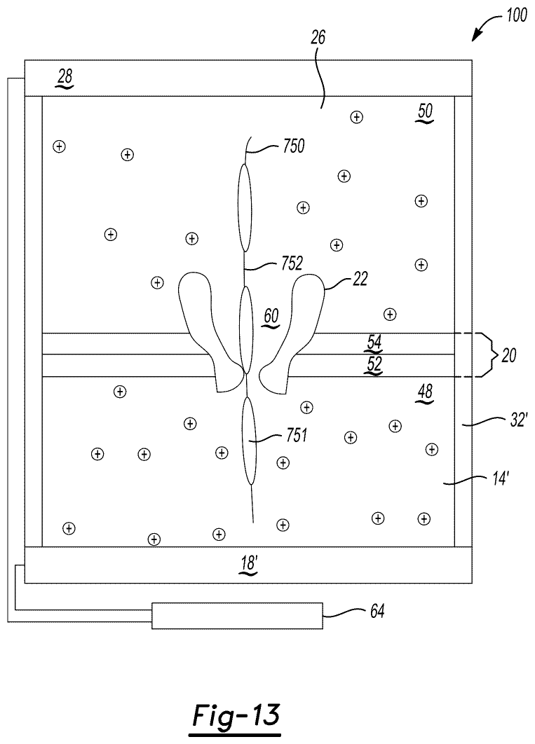

FIG. 13 schematically illustrates a cross-sectional view of another example use of the instrument of FIG. 2. As illustrated in FIG. 13, instrument 100 may include cis well 26, membrane 20 (which may have a configuration such as described with reference to FIG. 4A-4C, 8A-8B, or 9), first and second fluids 48, 50, and nanopore 22, all of which may be configured similarly as described with reference to FIG. 10. In the example illustrated in FIG. 13, surrogate polymer 750 is translocated through nanopore 22 under an applied force, e.g., a bias voltage that circuitry 64 applies between electrode 18′ and electrode 28. As used herein, a “surrogate polymer” is intended to mean an elongated chain of labels having a sequence corresponding to a sequence of nucleotides in a polynucleotide. In the example illustrated in FIG. 13, surrogate polymer 750 includes labels 751 coupled to one another via linkers 752. An XPANDOMER™ is a particular type of surrogate polymer developed by Roche Sequencing, Inc. (Pleasanton, CA). XPANDOMERS™ may be prepared using Sequencing By eXpansion™ (SBX™, Roche Sequencing, Pleasanton CA). In Sequencing by eXpansion™, an engineered polymerase polymerizes xNTPs which include nucleobases coupled to labels via linkers, using the sequence of a target polynucleotide. The polymerized nucleotides are then processed to generate an elongated chain of the labels, separated from one another by linkers which are coupled between the labels, and having a sequence that is complementary to that of the target polynucleotide. For example descriptions of XPANDOMERS™, linkers (tethers), labels, engineered polymerases, and methods for SBX™, see the following patents, the entire contents of each of which are incorporated by reference herein: U.S. Pat. Nos. 7,939,249, 8,324,360, 8,349,565, 8,586,301, 8,592,182, 9,670,526, 9,771,614, 9,920,386, 10,301,345, 10,457,979, 10,676,782, 10,745,685, 10,774,105, and 10,851,405.

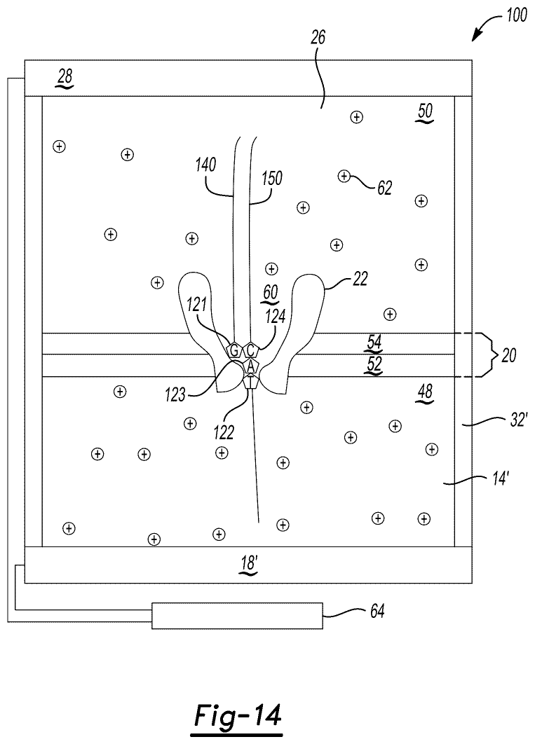

FIG. 14 schematically illustrates a cross-sectional view of another example use of the instrument of FIG. 2. As illustrated in FIG. 14, instrument 100 may include cis well 26, membrane 20 (which may have a configuration such as described with reference to FIG. 4A-4C, 8A-8B, or 9), first and second fluids 48, 50, and nanopore 22, all of which may be configured similarly as described with reference to FIG. 10. In the example illustrated in FIG. 14, a duplex between polynucleotide 140 and polynucleotide 150 is located within nanopore 22 under an applied force, e.g., a bias voltage that circuitry 64 applies between electrode 18′ and electrode 28. A combination of bases in the double-stranded portion (here, the base pair GC 121, 124 at the terminal end of the duplex) and bases in the single-stranded portion of polynucleotide 150 (here, bases A and T 123, 122) may alter the rate at which salt 62 moves through opening 60, and thus may detectably alter the electrical conductivity of opening 60 in such a manner as to be detected by circuitry 64. For further details regarding use of nanopores to sequence polynucleotides being translocated therethrough, see U.S. Patent Publication No. 2023/0090867 to Mandell et al., the entire contents of which are incorporated by reference herein.

To further illustrate the present disclosure, examples are given herein. It is to be understood that these examples are provided for illustrative purposes and are not to be construed as limiting the scope of the present disclosure.

### NON-LIMITING WORKING EXAMPLES

### Example 1

Four fluidic devices similar to FIG. 1 were prepared, except that the support structure of each included a plurality of wells, and a flow channel was formed over the wells. The apertures leading to the respective wells had 50 μm diameters.

Bubble painting was used to form the initial membranes. In this process, 1 μL of polybutadiene-polyethylene oxide block copolymer in octane between two air bubbles (2 μL each) was injected into each flow channel at a speed of 1 μL/s to generate a polymer bubble for painting.

After the membranes were initially prepared and before pore insertion, the membrane painting efficiency (%) and average capacitance (pF) were determined/measured. The membrane painting efficiency represents a percentage of membranes that were successfully formed with respect to all available membrane painting channels. Capacitance of each device was directly measured by the instrument (electrical stimulation using a triangular voltage waveform was applied and capacitive current is measured). The results are shown in Table 1. The results for the initial structures (column 1) represent the average results for all four of the fluidic devices.

MspA pores were introduced in a buffer solution (400 mM KCl, 50 mM HEPES buffer), and the fluidic devices were exposed to electrical pulses for pore insertion. The waveform that was used was made of a train of positive voltage pulses (+300 mV with a 10 ms duration) spaced by the reading steps: 140 ms at 0 mV bias and 300 ms at +50 mV (400 pulses in total). During a first cycle, the protocol was applied continuously for a period of 3 minutes and the magnitude of the pulses was kept at +300 mV. Further applied cycles (3 minutes each) were characterized by increasing of the pulsing voltage from 300 mV to 800 mV (50 mV step).

After the membranes were initially prepared and the nanopores were initially inserted in each fluidic device, the fluidic device was flushed with 1 mL of buffer at a rate of 5 μL/s to remove an excess of pores from the device. The electrical stability (V_{1/2}, 300 mV-1000 mV micropulses), pore insertion efficiency (%), and single pore conductance (pS) were then determined/measured.

The waveform that was used for the electrical stability test was made of a train of positive voltage pulses (+300 mV with a 10 us duration) spaced by 30 ms at +50 mV (each 20 pulse chunk was subsequently spaced by 100 ms of 100 mV bias). During the first cycle, the protocol was applied continuously for a period of 3 minutes and the magnitude of the pulses was kept at +300 mV. Further applied cycles (5 minutes each) were characterized by increasing the pulsing voltage from 300 mV to 1000 mV (at 100 mV steps). V½ corresponds to the voltage at which half of the membrane survived. Pore insertion efficiency is the percentage of the wells having a membrane with single pores from all available wells with membranes. The single pore conductance was measured by the instrument.

These results are also shown in Table 1. Again, the results for the initial structures (column 1) represent the average results for all four of the fluidic devices.

Each fluidic device was then exposed to electrical pulses (5 pulses of 1800 V at 2 ms each) that broke all of the membranes to form membrane fragments.

Different sized air bubbles were then introduced into the respective fluidic devices at different speeds to test the formation of membranes from the membrane fragments. After bubble introduction, the fluidic devices were exposed to electrical pulses for pore reinsertion in the same manner as described in this Example for the initial pore insertion.

After the membranes were repainted, the membrane painting efficiency (%) and average capacitance (pF) were measured as described in this Example. After the membranes were repainted and the nanopores reinserted, the fluidic device was flushed with 1 mL of buffer at a rate of 5 μL/s to remove an excess of pores from the device. The electrical stability (V_{1/2}, 300 mV-1000 mV micropulses), pore insertion efficiency (%), and single pore conductance (pS) were then determined/measured as described in this Example. The results for the individual fluidic devices are also shown in Table 1.

TABLE 1

| | Initial | | | | |
|---|---|---|---|---|---|
| | Structures | | | | |
| | (Averages | Reconfigured | Reconfigured | Reconfigured | Reconfigured |
| | for all 4 | Structure | Structure | Structure | Structure |
| | fluidic | 0.5 μL bubble | 4 μL bubble | 50 μL bubble | 4 μL bubble |
| | devices) | @ 1 μL/s | @ 1 μL/s | @ 1 μL/s | @ 5 μL/s |
| Membrane | 0.97 ± 0.07 | 0.97 | 1 | 0.71 | 0.97 |
| Painting | | | | | |
| Efficiency (%) | | | | | |
| Capacitance | 0.97 ± 0.2 | 0.7 ± 0.3 | 0.65 ± 0.2 | 0.72 ± 0.4 | 0.6 ± 0.2 |
| (pF) | | | | | |
| Electrical | 800 ± 100 | 800 | 900 | 800 | 900 |
| Stability (V_{1/2}) | | | | | |
| Pore | 0.65 ± 0.18 | 0.57 | 0.84 | 0.90 | 0.945 |
| Insertion | | | | | |
| Efficiency (%) | | | | | |
| S. Pore | 675 ± 25 | 669 ± 55 | 677 ± 24 | 640 ± 50 | 746 ± 25 |
| Conductance (%) | | | | | |

These results illustrate that the broken membranes and nanopores can be reconfigured in the fluidic device. The air bubble size had more of an impact on formation of the membranes from membrane fragments than air bubble speed. These results indicate that the air bubble size depends on the flow channel geometry. The air bubble should be large enough to fill the width of the channel and pass through all of the wells.

### Example 2

A fluidic device similar to FIG. 1 was prepared, except that the support structure of each included a plurality of wells, and a flow channel was formed over the wells. The apertures leading to the respective wells had 50 μm diameters.

A polybutadiene-polyethylene oxide block copolymer in octane was introduced into the flow channel and the corresponding wells, and bubble painting was used to form the initial membranes in the same manner described in Example 1. MspA pores were introduced in a buffer solution (400 mM KCl, 50 mM HEPES buffer), and allowed to incubate for 3 hours. This enabled the pores to non-specifically bind to the membranes, without being inserted into the membranes. The fluidic device was flushed with 1 mL of buffer at a rate of 5 μL/s to remove an excess of pores from the device. The fluidic device was then exposed to the same waveform described in Example 1 to break all of the membranes into membrane fragments.

An air bubble (4 μL) was then introduced into the fluidic device at a speed of 1 μL/s to test membrane formation from the membrane fragments. After bubble introduction, the fluidic device was exposed to the same waveform described in Example 1 for pore insertion.

After the membranes were painted from the membrane fragments and the nanopores were inserted, the fluidic device was flushed with 1 mL of buffer at a rate of 5 μL/s to remove an excess of pores from the device, and the pore insertion efficiency (%) was measured. In this example, the pore insertion efficiency was about 57%.

This example illustrates that a single pore insertion step may be performed after membranes are formed from membrane fragments in the fluidic device and in preparation of the fluidic device disclosed herein.

### Example 3

A fluidic device similar to FIG. 1 was prepared, except that the support structure of each included a plurality of wells, and a flow channel was formed over the wells. The apertures leading to the respective wells had 50 μm diameters.

A polybutadiene-polyethylene oxide block copolymer in octane was introduced into the flow channel and the corresponding wells, and bubble painting was used to form the initial membranes in the same manner described in Example 1. After the membranes were initially prepared and before pore insertion, the capacitance (pF) was measured. The results are shown in Table 2.

MspA pores were introduced in a buffer solution (400 mM KCl, 50 mM HEPES buffer), and the fluidic devices were exposed to the same waveform described in Example 1 for pore insertion. The fluidic device was then flushed with 1 mL of buffer at a rate of 5 μL/s to remove an excess of pores from the device. After the membranes were initially prepared, the nanopores initially inserted, and the washing step, the pore insertion efficiency (%) and single pore conductance (pS) were determined/measured as described in Example 1. These results are also shown in Table 2.

The fluidic device was exposed to the same waveform described in Example 1 to break all of the membranes into membrane fragments.

An air bubble (4 μL) was then introduced into the fluidic device at a speed of 1 μL/s to test membrane formation from the membrane fragments. After the membranes were painted from the membrane fragments, the capacitance was again measured. After bubble introduction, the fluidic device was exposed to the same waveform described in Example 1 for pore reinsertion. The fluidic device was then flushed with 1 mL of buffer at a rate of 5 μL/s to remove an excess of pores from the device. After the membranes were painted from the membrane fragments and the nanopores were reinserted in the fluidic device for the first time and the washing step was performed, the pore insertion efficiency (%) and single pore conductance (pS) were again determined/measured. These results for the fluidic device are also shown in Table 2.

The reconfigured fluidic device was again exposed to the same waveform described in Example 1 to break the repainted membranes into membrane fragments.

An air bubble (4 μL) was then introduced into the fluidic device at a speed of 1 μL/s to test membrane formation from the membrane fragments. After the membranes were painted from the membrane fragments, the capacitance was again measured. After bubble introduction, the fluidic device was exposed to the same waveform described in Example 1 for pore insertion. The fluidic device was flushed with 1 mL of buffer at a rate of 5 μL/s to remove an excess of pores from the device. After the membranes were painted from the membrane fragments and the nanopores were reinserted in the fluidic device for the second time and the washing step was performed, the pore insertion efficiency (%) and single pore conductance (pS) were again determined/measured. These results for the fluidic device also shown in Table 2.

TABLE 2

| | | Structure | Structure |
|---|---|---|---|
| | Initial | Reconfigured | Reconfigured |
| | Structure | First time | Second time |
| Capacitance | 0.8 ± 0.1 | 0.9 ± 0.1 | 0.2 ± 0.1 |
| (pF) | | | |
| Pore | 82.8 | 73.9 | 55.1 |
| Insertion | | | |
| Efficiency | | | |
| (% of wells | | | |
| with inserted | | | |
| pores) | | | |
| S. Pore | 675 ± 18 | 691 ± 44 | 697 ± 54 |
| Conductance | | | |
| (%) | | | |

These results illustrate that the broken membranes and bound nanopores can be reconfigured in the fluidic device multiple times. While the results for pore insertion efficiency for the second reconfiguration are lower than the first reconfiguration, it is believed that the introduction of additional membrane material during the second reconfiguration may improve these results.

### Example 4

A fluidic device similar to FIG. 1 was prepared, except that the support structure of each included a plurality of wells, and a flow channel was formed over the wells. The apertures leading to the respective wells had 16 μm diameters.

Bubble painting was used to form the initial membranes. In this process, 0.5 μL of polybutadiene-polyethylene oxide block copolymer in octane between two air bubbles (2 μL each) was injected into the flow channel at a speed of 1 μL/s to generate a polymer bubble for painting. After the membranes were initially prepared and before pore insertion, the membrane painting efficiency (%) and the capacitance (pF) were determined/measured. These results are shown in Table 3.

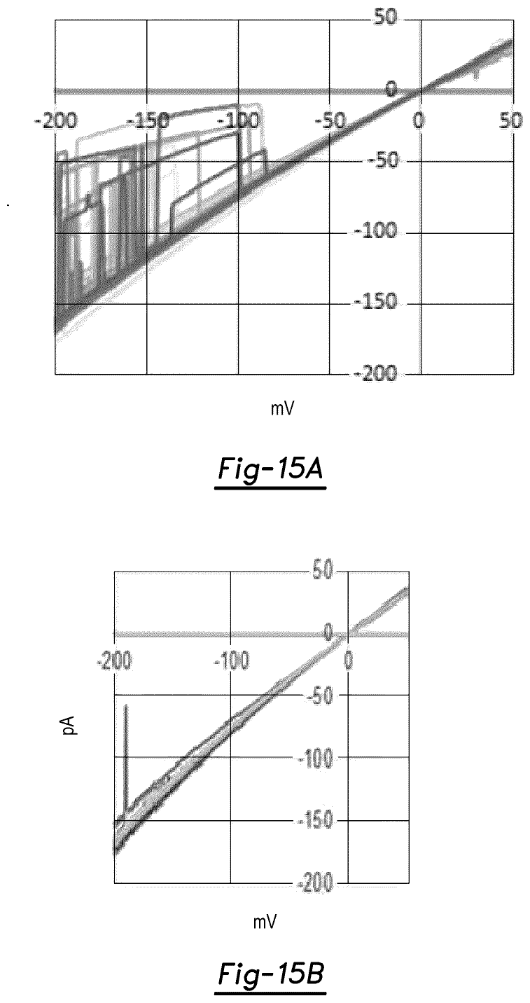

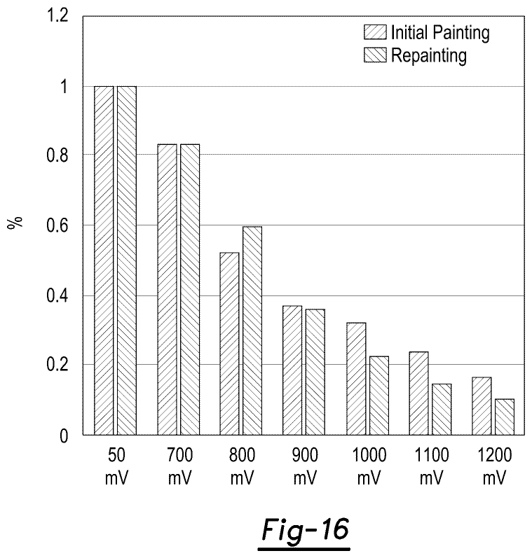

MspA pores were introduced in a buffer solution (400 mM KCl, 50 mM HEPES buffer), and the fluidic device was exposed to the same waveform described in Example 1 for pore insertion. The fluidic device was flushed with 1 mL of buffer at a rate of 5 μL/s to remove an excess of pores from the device. After the membranes were initially prepared and the nanopores were initially inserted and the washing step was performed, the capacitance (pF), pore insertion efficiency (%), and single pore IV (current (pA) vs. voltage (mV)) curves were determined/measured. The capacitance and pore insertion efficiency results are shown in Table 3. The I/V curves represent the relationship between the applied voltage and the current through a single pore that was measured during application of voltage bias. These results are shown in FIG. 15A.

The fluidic device was exposed to the same waveform described in Example 1 to break the membranes into membrane fragments.

An air bubble (4 μL) was then introduced into the fluidic device at a speed of 1 μL/s to test membrane formation from the membrane fragments. After the membranes were painted from the membrane fragments, the membrane painting efficiency and capacitance were again determined/measured. After bubble introduction, the fluidic device was exposed to the same waveform described in Example 1 for pore reinsertion. The fluidic device was flushed with 1 mL of buffer at a rate of 5 μL/s to remove an excess of pores from the device. After the membranes were repainted and the nanopores were reinserted and the washing step was performed, the pore insertion efficiency (%) and single pore IV (current (pA) vs. voltage (mV)) curves were determined/measured again. These results for the fluidic device are shown in Table 3 and FIG. 15B, respectively.

TABLE 3

| | Initial | Reconfigured |
|---|---|---|
| | Structure | Structure |
| Membrane | 100 | 60 |
| Painting | | |
| Efficiency | | |
| (%) | | |
| Capacitance | 0.4 ± 0.1 | 0.4 ± 0.1 |
| (pF) | | |
| Pore | 88 | 57 |
| Insertion | | |
| Efficiency | | |
| (%) | | |

This example illustrates that the membrane repainting and nanopore insertion/reinsertion methods described herein are suitable for use on a variety of fluidic device designs.

In this example, it was noted that the pores exhibited less gating at negative bias after membrane reconfiguration, which can be beneficial for recharging electrodes of the fluidic device.

### Example 5

A fluidic device similar to FIG. 1 was prepared, except that the support structure of each included a plurality of wells, and a flow channel was formed over the wells. The apertures leading to the respective wells had 50 μm diameters.

A polybutadiene-polyethylene oxide block copolymer in octane was introduced into the flow channel and the corresponding wells, and bubble painting was used to form the initial membranes in the same manner described in Example 1.

MspA pores were introduced in a buffer solution 400 mM KCl, 50 mM HEPES buffer), and pore insertion was controlled manually. After the introduction of the pores, pulses (200 ms duration) with increasing voltage (200-250-300-350-400-425 mV) were performed. Each voltage step took approximately 2 minutes to 5 minutes and frequency was ˜1 pulse/s. The fluidic device was flushed with 1 mL of buffer at a rate of 5 μL/s to remove an excess of pores from the device.

The pore insertion efficiency (%) was then determined, and the results are shown in Table 4. The nanopore instrument was exposed to an electrical pulse(s) (1-3 pulses of 1.5 V) that broke at least some of the membranes into membrane fragments. An air bubble (4 μL) was then introduced into the fluidic device at a speed of 1 μL/s to test membrane formation from the membrane fragments. After bubble introduction, the instrument was exposed to the same manual pore insertion technique described in this Example. The fluidic device was flushed with 1 mL of buffer at a rate of 5 μL/s to remove an excess of pores from the device. After the membranes were formed from the membrane fragments and the nanopores were reinserted and the washing step was performed, the pore insertion efficiency was again determined. These results are also shown in Table 4.

Electrical stress (waveform: 360 ms of −50 mV, 50 ms of +40 mV, 50 ms of +60 mV and 50 ms of +80 mV) was applied to the reconfigured structures. The pore stability was determined after 24 hours of application of the electrical stress waveform. These results are also shown in Table 4.

TABLE 4

| | | | Reconfigured |
|---|---|---|---|
| | | | Structure |
| | | | After 24 Hrs. |
| | Initial | Reconfigured | electrical |
| | Structure | Structure | stress |
| % of wells with single | 81 | 62 | 62 |
| pores | | | |

These results illustrate that prolonged application of electrical waveform used for sequencing does not impact the stability of the reinserted nanopores.

### Example 6

A fluidic device similar to FIG. 1 was prepared, except that the support structure of each included a plurality of wells, and a flow channel was formed over the wells. The apertures leading to the respective wells had 50 μm diameters.

A polybutadiene-polyethylene oxide block copolymer in octane was introduced into the flow channel and the corresponding wells, and bubble painting was used to form the initial membranes in the same manner described in Example 1.

MspA pores were introduced in a buffer solution (400 mM KCl, 50 mM HEPES buffer), and the fluidic devices were exposed to the same waveform described in Example 1 for pore insertion. The fluidic device was flushed with 1 mL of buffer at a rate of 5 μL/s to remove an excess of pores from the device.

To assess the stability of the initially inserted pores, stepwise increased high voltage micropulses were applied. The percentage of inserted pores was initially detected at a bias of +50 mV, and then the high voltage micropulses were applied. The electrical waveform applied in this Example was made of a train of positive voltage pulses (+700 mV with a 10 us duration) spaced by 30 ms at +50 mV (each 20 pulse chunk was subsequently spaced by 100 ms of −100 mV bias). During the first cycle, the protocol was applied continuously for a period of 5 minutes and the magnitude of the pulses was kept at +700 mV. Further applied cycles (5 minutes each) were characterized by increasing the pulsing voltage from 700 mV to 1200 mV (in 100 mV steps). The percentage of inserted pores was detected at various biases throughout the test. The results are shown in FIG. 16 as “initial painting.”

The initially formed membranes were then broken and repainted as described in Example 1. Pore reinsertion was performed as follows.

The waveform that was used was made of a train of positive voltage pulses (+300 mV with a 10 ms duration) spaced by the reading steps: 140 ms at 0 mV bias and 300 ms at +50 mV (400 pulses in total). During a first cycle, the protocol was applied continuously for a period of 3 minutes and the magnitude of the pulses was kept at +300 mV. Further applied cycles (3 minutes each) were characterized by increasing of the pulsing voltage from 300 mV to 800 mV (50 mV step). The fluidic device was flushed with 1 mL of buffer at a rate of 5 μL/s to remove an excess of pores from the device. The percentage of reinserted pores was detected at various biases throughout the test. The results are shown in FIG. 16 as “repainting.”

The results in FIG. 16 illustrate that the repainted membranes and reinserted pores exhibit comparable stability to the initially painted membranes and inserted pores.

### Example 7

Four fluidic devices were prepared in the same manner as Example 1. For each of these fluidic devices, initial membranes were painted as described in Example 1 and pores were inserted into the initially formed membranes as described in Example 1. The fluidic devices were flushed with 1 mL of buffer at a rate of 5 μL/s to remove an excess of pores from the devices.

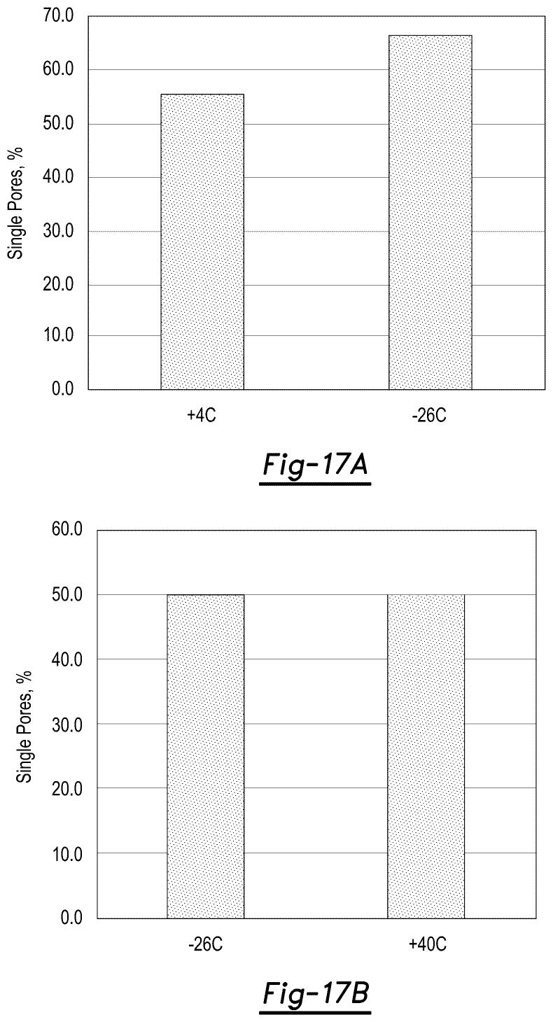

The first and second fluidic devices were exposed to temperature testing. The membranes of the first fluidic device were broken into membrane fragments (as described in Example 1), and the first fluidic device was stored in a refrigerator (at 4° C.) overnight. The membrane fragments were then repainted to form membranes and the nanopores were reinserted as described in Example 2. The pore insertion efficiency was determined after refrigeration, repainting, and reinsertion and these results are shown in FIG. 17A at “+4 C.” The membranes of the first fluidic chip were then broken again (as described in Example 1), and the first fluidic device was stored in a freezer (at −26° C.) overnight. The membrane fragments were then repainted to form membranes and the nanopores were reinserted as described in Example 2. The first fluidic device was flushed with 1 mL of buffer at a rate of 5 μL/s to remove an excess of pores from the device. The pore insertion efficiency was determined after freezing, repainting, and reinsertion and these results are shown in FIG. 17A at “−26 C.”

The membranes of the second fluidic device were broken into membrane fragments (as described in Example 1), and the second fluidic device was stored in a freezer (at −26° C.) overnight. The membrane fragments were then repainted to form membranes and the nanopores were reinserted as described in Example 2. The second fluidic device was flushed with 1 mL of buffer at a rate of 5 μL/s to remove an excess of pores from the device. The pore insertion efficiency was determined after freezing, repainting, and reinsertion and these results are shown in FIG. 17B at “−26 C.” The membranes of the second fluidic chip were then broken again (as described in Example 1), and the second fluidic device was put on a heating plate (at +40° C.) overnight. The membrane fragments were then repainted to form membranes and the nanopores were reinserted as described in Example 2. The second fluidic device was again flushed with 1 mL of buffer at a rate of 5 μL/s to remove an excess of pores from the device. The pore insertion efficiency was determined after heating, repainting, and reinsertion and these results are shown in FIG. 17B at “+40 C.”

The results in FIG. 17A and FIG. 17B illustrate that the fluidic devices in the inoperative positions (i.e., membrane fragments over interstitial regions) can be stored at and moved between various temperatures, and that the extreme temperature exposure did not deleteriously affect the efficiency of pore reinsertion.

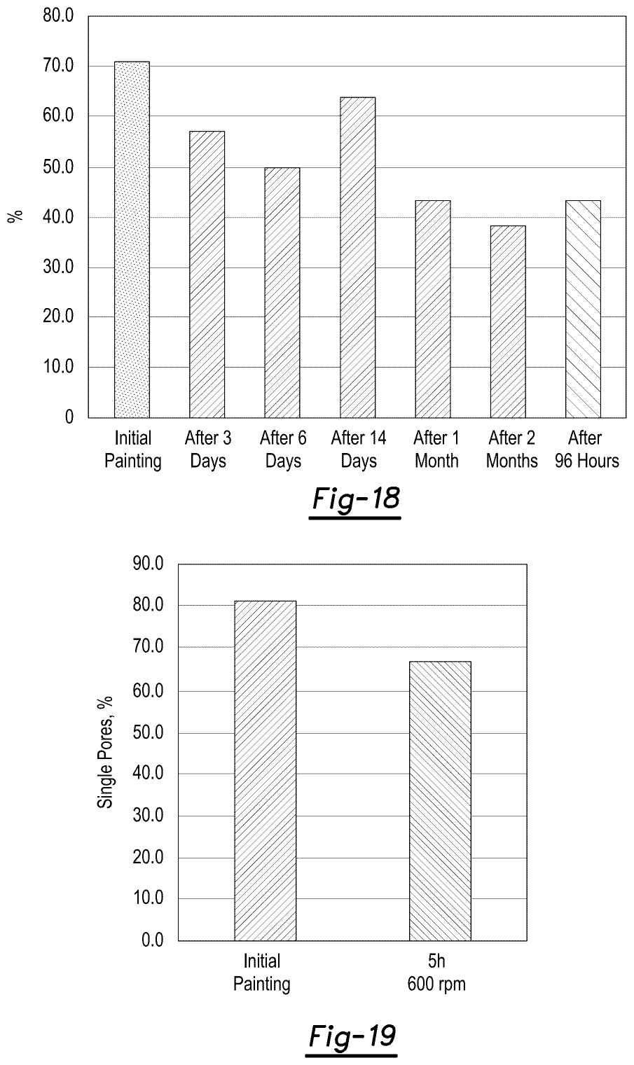

The third fluidic device was exposed to time testing. After initial membrane painting and pore insertion (as described in Example 1), the third fluidic device again flushed with 1 mL of buffer at a rate of 5 μL/s to remove an excess of pores from the device, and the pore insertion efficiency was determined. These results are shown in FIG. 18 as “initial painting.” The third fluidic device was then exposed to several cycles of breaking (Example 1), repainting (Example 2), pore reinsertion (Example 1), and washing performed as follows: 3 days after initial painting, 6 days after initial painting, 14 days after initial painting, 1 month after initial painting, and 2 months after initial painting.

The pore insertion efficiency was determined after each cycle. These results are also shown in FIG. 18 and are identified by the cycle period. The third fluidic device was then exposed to an electrical stability test for 96 hours as described in Example 5. These results are also shown in FIG. 18 and are identified as “after 96 hours.” These results indicate that the fluidic device remains relatively stable for at least two months.

The fourth fluidic device was exposed to mechanical testing. After initial membrane painting and pore insertion and washing as described herein, the pore insertion efficiency was determined. These results are shown in FIG. 19 as “initial painting.” The fourth fluidic device was then exposed to stress on a shaking plate at 600 rpm for 5 hours. The membrane fragments were then repainted to form membranes and the nanopores were reinserted as described in Example 2. The fourth fluidic device was again flushed with 1 mL of buffer at a rate of 5 μL/s to remove an excess of pores from the device. The pore insertion efficiency was determined and these results are also shown in FIG. 19. These results indicate that the fluidic device in the inoperative position can be reconfigured with a desirable number of inserted pores even after severe mechanical stress.

### Example 8

Another fluidic device was prepared in the same manner as Example 1. The initial membranes were painted and pores were inserted into the initially formed membranes and the device was washed as described in Example 1. The membranes were also broken as described in Example 1. The membranes were repainted as described in Example 2 and the nanopores were reinserted as described in Example 1 and the device was washed again as described in Example 1.

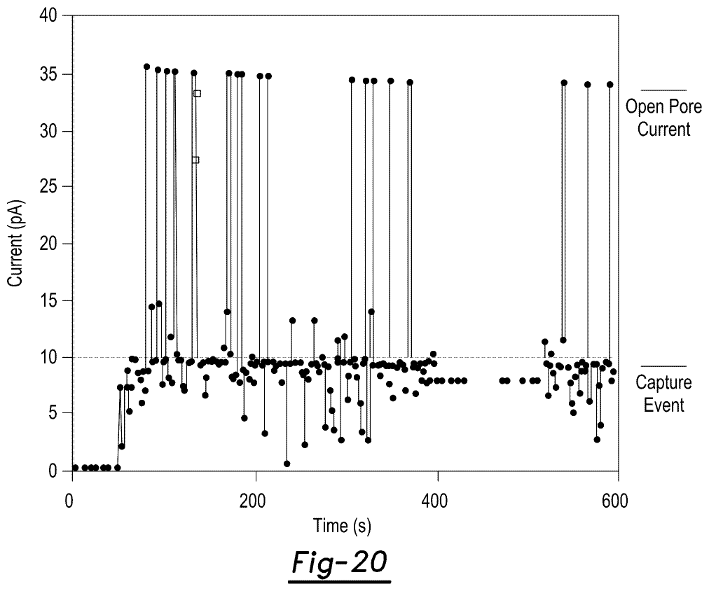

DNA was introduced into the channel and the capture rate of the DNA was controlled by the pore. FIG. 20 depicts the capture rate results for one of the pores. At about 35 pA, the nanopore is open, and at about 10 pA, DNA blocks the current. The latter is recognized as a capture event. The waveform used in this Example was as follows: 600 ms of +140 mV, 1300 ms of +50 mV, 100 ms of −80 mV. The capture rate of the reconfigured fluidic structure was about 69%, illustrating that the reconfigured fluidic device is suitable for sequencing.

### Additional Comments

The example fluidic devices and nanopore instruments disclosed herein exhibit mechanical stability, relatively long shelf lives, and stability over a relatively broad temperature range.

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

What is claimed is:

1. A fluidic device, comprising:
a support structure defining a well and an interstitial region surrounding the well;
an electrode operatively positioned at a bottom of the well;
membrane fragments bound over the interstitial region; and
a nanopore, a nanopore subunit, or a combination thereof bound over the interstitial region.

2. The fluidic device as defined in claim 1, wherein:
the membrane fragments are a polymeric membrane material; and
the nanopore is a biological nanopore or a hybrid nanopore.

3. The fluidic device as defined in claim 1, wherein the support structure includes a substrate and a patterned layer over the substrate.

4. A nanopore instrument, comprising:
a support structure defining a plurality of trans wells and interstitial regions surrounding each of the plurality of trans wells;
a trans electrode associated with the trans wells;
a cis well in fluid communication with at least some of the plurality of trans wells;
a cis electrode associated with the cis well;
membrane fragments bound over the interstitial regions; and
a nanopore, a nanopore subunit, or a combination thereof bound over the interstitial regions.

5. The nanopore instrument as defined in claim 4, wherein:
the membrane is a polymeric membrane; and
the nanopore is a biological nanopore or a hybrid nanopore.

6. The nanopore instrument as defined in claim 4, wherein the nanopore instrument includes a plurality of the trans electrodes, and wherein each of the plurality of trans electrodes is respectively associated with one of the plurality of trans wells.

7. The nanopore instrument as defined in claim 6, further comprising:
a stimulus source coupled to each of the plurality of trans electrodes either individually or via multiplexing; and
a controller coupled to the stimulus source, the controller configured to individually/selectively address the plurality of trans electrodes.

8. The nanopore instrument as defined in claim 4, wherein the plurality of trans wells and the cis well are free of a liquid.

9. The nanopore instrument as defined in claim 4, wherein the plurality of trans wells and the cis well contain a buffer.

10. The nanopore instrument as defined in claim 9, wherein the buffer is an electrode buffer.

11. A method, comprising:
introducing a gas bubble into a flow through channel of a fluidic device including:
a support structure defining a well and an interstitial region surrounding the well;
the flow through channel in fluid communication with the well and the interstitial region;
an electrode operatively positioned at a bottom of the well;
membrane fragments bound over the interstitial region; and
a nanopore, a nanopore subunit, or a combination thereof bound over the interstitial region;
wherein introducing the gas bubble causes the membrane fragments to forma membrane across an aperture leading to the well; and
applying a voltage to the electrode, thereby causing insertion of the nanopore or a reassembled nanopore into the formed membrane.

12. The method as defined in claim 11, wherein:
the well is one of a plurality of trans wells;
the interstitial region surrounds each of the plurality of trans wells;
the flow through channel is in fluid communication with each of the plurality of trans wells; and
introducing the gas bubble into the flow through channel involves introducing the gas bubble into an inlet and guiding the gas bubble from the inlet through the flow through channel in a single direction to an outlet, thereby causing the membrane fragments to form respective membranes across respective apertures of at least some of the plurality of trans wells.

13. The method as defined in claim 12, wherein:
a plurality of the nanopore is bound over the interstitial region;
respective electrodes are positioned at the bottom of each of the plurality of trans wells; and
the voltage is applied as voltage pulses to each of the respective electrodes, thereby inserting one of the plurality of the nanopores into each of the respective membranes.

14. The method as defined in claim 11, further comprising introducing additional membrane material with the gas bubble.

15. A method, comprising:
forming a membrane across an aperture of a well from membrane fragments bound over an interstitial region surrounding the well by:
introducing a hydrophobic liquid into the flow through channel of a fluidic device including:
a support structure defining the well and the interstitial region;
the flow through channel in fluid communication with the well and the interstitial region;
an electrode operatively positioned at a bottom of the well;
the membrane fragments bound over the interstitial region;
a nanopore, a nanopore subunit, or a combination thereof bound over the interstitial region; and
an aqueous liquid contained in the well and in the flow through channel;
while the hydrophobic liquid is present in the flow through channel, flowing a second aqueous liquid through the flow through channel; and
applying a voltage to the electrode, thereby causing insertion of the nanopore or a reassembled nanopore into the formed membrane.

16. The method as defined in claim 15, wherein:
the well is one of a plurality of trans wells;
the interstitial region surrounds each of the plurality of trans wells;
the flow through channel is in fluid communication with each of the plurality of trans wells; and
flowing the second aqueous liquid through the flow through channel involves introducing the second aqueous liquid into an inlet and guiding the second aqueous liquid from the inlet through the flow through channel in a single direction to an outlet, thereby causing the membrane fragments to form respective membranes across respective apertures of at least some of the plurality of trans wells.

17. The method as defined in claim 16, wherein:
a plurality of the nanopore is bound over the interstitial region;
respective electrodes are positioned at the bottom of each of the plurality of trans wells; and
the voltage is applied as voltage pulses to each of the respective electrodes, thereby inserting at least some of the plurality of the nanopores into at least some of the respective membranes.

18. The method as defined in claim 15, further comprising introducing additional membrane material with the hydrophobic liquid.

19. A method, comprising:
forming a membrane across an aperture of a well defined by a support structure, wherein the membrane is supported by an interstitial region of the support structure;
inserting a nanopore into the membrane; and
breaking the membrane into membrane fragments that become bound over the interstitial region, wherein the nanopore or subunits thereof non-specifically binds over the interstitial region.

20. The method as defined in claim 19, wherein breaking the membrane involves exposing the membrane to a voltage ranging from about 1 V to about 2 V.

21. The method as defined in claim 19, wherein breaking the membrane involves exposing the membrane to a surfactant.

22. The method as defined in claim 19, wherein breaking the membrane involves exposing the membrane to osmotic pressure.

23. The method as defined in claim 19, wherein breaking the membrane involves exposing the membrane to high velocity buffer flow.
